(19) United States
(12) Patent Application Publication
(10) Pub. No.: US 2022/0351530 A1
(43) Pub. Date: Nov. 3, 2022

(54) SYSTEM AND METHOD OF SCREENING BIOLOGICAL OR BIOMEDICAL SPECIMENS

(71) Applicants: B.G. Negev Technologies & Applications Ltd., at Ben-Gurion University, Beer-Sheva (IL); The Board of Regents of The University of Texas System, Austin, TX (US)

(72) Inventors: Assaf ZARITSKY, Lehavim (IL); Gaudenz DANUSER, Dallas, TX (US); Andrew R. JAMIESON, McKinney, TX (US)

(21) Appl. No.: 17/735,344

(22) Filed: May 3, 2022

Related U.S. Application Data
(60) Provisional application No. 63/183,072, filed on May 3, 2021.

(51) Int. Cl. G06V 20/69; G01N 33/483; G06V 10/776; G06T 7/00

(52) U.S. Cl. CPC G06V 20/698; G01N 33/4833; G06V 20/695; G06V 10/776; G06T 7/0012; G06T 2207/20081

(57) ABSTRACT

A system and method of screening biological specimens by at least one processor may include receiving a sample image depicting a biological specimen; applying a machine-learning (ML) based autoencoder on the sample image, wherein said autoencoder is trained to generate a reconstructed version of the sample image, via a latent feature vector; associating a latent feature of the latent feature vector to a corresponding visual phenotype of the biological specimen; and screening the biological specimen based on said association. Embodiments of the invention may subsequently modify a value of the latent feature to produce a vector set, comprising a plurality of latent feature vectors; apply a decoder portion of the autoencoder on the vector set, to produce a corresponding reconstructed image set, representing evolution or amplification of a visual phenotype of the biological specimen; and associate the latent feature to the visual phenotype based on the reconstructed image set.

### CROSS-REFERENCE TO RELATED APPLICATIONS

This application claims the benefit of priority of U.S. Provisional Patent Application No. 63/183,072, filed May 3, 2021, entitled “INTERPRETABLE DEEP LEARNING UNCOVERS CELLULAR PROPERTIES IN LABEL-FREE LIVE CELL IMAGES THAT ARE PREDICTIVE OF HIGHLY-METASTATIC MELANOMA”, the contents of which are all incorporated herein by reference in their entirety.

### FIELD OF THE INVENTION

The present invention relates generally to the field of assistive diagnostics. More specifically, the present invention relates to systems and methods of screening biological or biomedical specimens.

### BACKGROUND OF THE INVENTION

A neural network (NN) or an artificial neural network (ANN), e.g., a neural network implementing a machine learning (ML) or artificial intelligence (AI) function, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

Deep Learning Artificial Neural Networks have revolutionized machine learning and computer vision as powerful tools for complex pattern recognition, but there is increasing mistrust in results produced by ‘black-box’ neural networks. Aside from increasing the confidence, the interpretation of the properties—also referred to as ‘mechanisms’—of the pattern recognition process can potentially generate insight of a biological/physical phenomenon that escapes the analysis driven by human intuition.

In medical imaging the quest for interpretability has been responded by identifying image sub-regions of special importance for trained deep neural networks. A similar idea was implemented in fluorescent microscopy images, in the context of classification of protein subcellular localization, to visualize the supervised network activation patterns.

Localization of sub-regions that were particularly important for the classifier result permitted a visual assessment and pathological interpretation of distinctive image properties. Such approaches are only suitable when the classification-driving information is localized in one image region over another, and when highlighting the region is sufficient to establish a biological hypothesis.

### SUMMARY OF THE INVENTION

For cellular phenotyping, this is not the case. Because of the orthogonalization of feature space construction and classifier training we could elegantly extract visual cues for the inspection of classifier-relevant cell appearances. By exploiting the single cell variation of the latent feature space occupancy and the associated variation in the scoring of a classifier discriminating high from low metastatic melanoma, we identified latent features as predominant in prescribing metastatic propensity.

Embodiments of the invention may include a method of screening biological specimens by at least one processor. Embodiments of the method may include: receiving a sample image depicting a biological specimen; applying a machine-learning (ML) based autoencoder model on the sample image, wherein said autoencoder model may be trained to generate a reconstructed version of the sample image, via a latent feature vector; associating at least one latent feature of the latent feature vector to at least one corresponding visual phenotype of the biological specimen; and screening the biological specimen based on said association.

Additionally, or alternatively, embodiments of the invention may include, for at least one latent feature: modifying a value of the latent feature to produce a vector set, may include a plurality of latent feature vectors; applying a decoder portion of the autoencoder on the vector set, to produce a corresponding reconstructed image set, representing evolution of a visual phenotype of the biological specimen; and associating the latent feature to the visual phenotype based on the reconstructed image set.

Additionally, or alternatively, embodiments of the invention may include, for at least one latent feature: applying a first ML-based classification model on one or more latent feature vectors of the vector set, to calculate corresponding classification scores, wherein each classification score represents pertinence of a relevant latent feature vector to a predefined class; and associating the latent feature to the visual phenotype further based on the one or more classification scores.

Additionally, or alternatively, embodiments of the invention may include, for at least one latent feature: attributing a significance score to the at least one latent feature based on the classification scores, wherein said significance score represents significance of the at least one latent feature in driving an outcome of the first ML-based classification model; and associating the latent feature to the visual phenotype further based on the significance score.

According to some embodiments, modifying a value of at least one latent feature may include: for one or more latent features, determining a range and a step size, so as to define a latent feature space of the autoencoder model; and modifying the value of the one or more latent features so as to traverse through the latent space of the autoencoder model.

Embodiments of the invention may include selecting one or more latent features based on their attributed significance scores; calculating a trajectory that follows a gradient of the classification score based on the selected one or more latent features; and modifying the value of the one or more selected latent features according to the calculated trajectory, so as to traverse through a latent space of the autoencoder model.

Additionally, or alternatively, modifying a value of at least one latent feature may include: accumulating a plurality of original values of the latent feature, corresponding to a plurality of sample images; calculating a natural variation of a latent feature space, defined by the latent feature vector; and modifying the value of the latent feature beyond the calculated natural variation of the latent feature space.

Embodiments of the invention may include applying the autoencoder model on a sample image depicting a biological specimen, to produce a corresponding reconstructed version of the sample image; calculating a first loss function value, based on comparison between the sample image and reconstructed image; and training the autoencoder model so as to minimize said first loss function value.

Additionally, or alternatively, embodiments of the invention may include providing a second ML-based classification model, trained to calculate a classification score that represents pertinence of a biological specimen depicted in an image to a predefined class; applying the second classification model on the sample image to produce a first classification score; applying the second classification model on the reconstructed version of the sample image to produce a second classification score; and training the autoencoder model further based on the first classification score and second classification score.

Additionally, or alternatively, embodiments of the invention may include producing a second loss function value, representing discrepancy between a classification score of the sample image and a classification score of the reconstructed image; and training the autoencoder model further based on the second loss function value.

According to some embodiments, screening a biological specimen may be selected from a list consisting of providing diagnostic evaluation of an underlying cause that contributes the classification score, based on the visual phenotype of the biological specimen; performing triage of a biological specimen, based on a visual phenotype of the biological specimen; and applying a treatment agent, based on the visual phenotype of the biological specimen.

Embodiments of the invention may include a system for screening biological specimens by at least one processor. Embodiments of the system may include a non-transitory memory device, wherein modules of instruction code may be stored, and a processor associated with the memory device, and configured to execute the modules of instruction code. Upon execution of the modules of instruction code, the at least one processor may be configured to: receive a sample image depicting a biological specimen; apply an ML based autoencoder model on the sample image, wherein said autoencoder model may be trained to generate a reconstructed version of the sample image, via a latent feature vector; associate at least one latent feature of the latent feature vector to at least one corresponding visual phenotype of the biological specimen; and screen the biological specimen based on said association.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings.

FIG. P1 is a block diagram, depicting a computing device which may be included in a system for screening biological specimens, according to some embodiments of the invention.

FIG. P2 is a block diagram depicting a system for screening biological specimens, according to some embodiments of the invention.

FIG. P3 is a flow diagram depicting a method of screening biological specimens, according to some embodiments of the invention.

FIG. 1 depicts unsupervised learning of a latent vector that encodes characteristic features of individual melanoma cells. (A) Top: Snapshot of a representative field of view of m481 PDX cells. Scale bar=50 μm. Bottom: Time-lapse sequence of a single cell undergoing dynamic blebbing. Scale bar=50 μm. (B) Representative time-lapse images of single cells from PDX tumors exhibiting low (m498) and high (m634) metastatic efficiency. Sequential images were each acquired 1 minute apart. (C) Design of the adversarial autoencoder, comprising an encoder (dark red) to extract from single cell images a 56-dimensional latent vector, so that a decoder can reconstruct from the vector a similar image. The “adversarial” component (top) penalizes randomly generated latent cell descriptors q(z) that the network fails to distinguish from latent cell descriptors drawn from the distribution of observed cells p(z). (D) Examples of cell reconstructions. Raw cell images (top): beginning of epoch #110K (trained on 10,000 images), around midway training of epoch #11M (after 1,000,000 images), at the end of epoch #3, epoch #6, and epoch #46. (E) Convergence of autoencoder loss (binary cross-entropy between raw and reconstructed image). Epoch is a full data set training cycle that consists of ˜1.7 million images. Mini-batch is the number of images processed on the GPU at a time. Each mini-batch includes 50 cell images randomly selected for each network parameter learning update. For every epoch, the images order is scrambled and then partitioned into ordered sets of 50 for each mini-batch.

FIG. 2 depicts discrimination of different melanoma cell categories: melanoma cell line versus melanocytes (B-D), cell lines versus clonal expanded cell lines (E-G), and cell lines versus PDXs (H-J). (A) Blinding the cell type. A cell type was defined as a specific cell line or PDX. Categories encompass multiple cell types. Multiple rounds of training and testing were performed. In each round, data from one cell type was used as the test dataset, defining a single observation that was composed of many single cell classifications. The training set contained the rest of the data relevant for the task (e.g., all melanoma cell lines and all PDXs when discriminating these two categories). The trained model was completely blind to the cell type used in each test set. The trained model classified each single cell in the test set. (B) Receiver-Operator Characteristic (ROC) curve for the distinction of the category ‘cell lines’ from the category ‘melanocytes’. AUC=0.635. (C) Accuracy in predicting for a cell type its association with the category ‘cell lines’ versus the category ‘melanocytes’. Each data point indicates the outcome of testing a particular cell type by the fraction of individual cells classified as ‘cell line’. N=8 cell types: 6 melanoma cell lines, 2 melanocyte lines. 7/8 successful predictions. Wilcoxon rank-sum and Binomial statistical tests on the null hypothesis that the classifier scores of a cell line and of melanocytes are drawn from the same distribution, p=0.071 (Wilcoxon), p=0.035 (Binomial). (D) Bootstrap distribution of the prediction of a cell type as a member of the ‘cell lines’ category. For each cell type, we generated 1000 observations by repeatedly selecting 20 random cells and recorded the fraction of these cells that were classified as ‘cell lines’. Horizontal line — median. Wilcoxon rank-sum test p<0.0001 rejecting the null hypothesis that the classifiers scores of observations from the two categories stem from the same distribution. This analysis demonstrated the ability to discriminate cell lines versus melanocytes from random samples of 20 cells in a cell type. (E) ROC curve for the distinction of the category ‘cell lines’ from the category ‘clonal’ (expansion line). (F) Accuracy in predicting for a cell type its association with the category ‘cell lines’ versus the category ‘clonal’. Each data point indicates the outcome of testing a particular cell type by the fraction of individual cells classified as ‘cell line’. N=10 cell types: 6 melanoma cell lines, 4 clonal expansion lines. 10/10 successful predictions. Wilcoxon rank-sum and Binomial statistical test on the null hypothesis that the classifier scores of a cell line and of a clonal expansion line are drawn from the same distribution, p=0.010 (Wilcoxon), p<0.001 (Binomial). (G) Bootstrap distribution of the prediction of a cell type as a member of the ‘cell lines’ category. See panel D. Horizontal line—median. Wilcoxon rank-sum test p<0.0001 rejecting the null hypothesis that the classifiers scores of observations from the two categories stem from the same distribution. (H) ROC curve for the distinction of the category ‘cell lines’ from the category ‘PDXs’. AUC=0.714. (I) Accuracy in predicting for a cell type its association with the category ‘cell lines’ versus the category ‘PDXs’. Each data point indicates the outcome of testing a particular cell type by the fraction of individual cells classified as ‘cell line’. N=15 cell types: 6 cells lines, 9 PDXs. 14/15 successful predictions. Wilcoxon rank-sum and Binomial statistical test on the null hypothesis that the classifier scores of cell lines and of PDX are drawn from the same distribution, p<0.0004 (Wilcoxon), p<0.0005 (Binomial). (J) Bootstrap distribution of the prediction of a cell type as a member of the ‘cell lines’ category. See panel D. Horizontal line—median. Wilcoxon rank-sum test p<0.0001 rejecting the null hypothesis that the classifiers scores of observations from the two categories stem from the same distribution. For all panels we used the time-averaged latent space vector over the entire movie as a cell's descriptor.

FIG. 3 depicts discrimination of PDXs with low versus high metastatic efficiency as defined by the correlation between outcomes in mouse and man (A). Classifiers were trained to predict metastatic efficiency at the single cell level (panels B, E). The association of a particular PDX with either the category ‘Low’ [metastatic efficiency] or the category ‘High’ [metastatic efficiency] was determined at the population level—either considering the fraction of all cells of a PDX predicted as ‘Low’ (C, F) or a bootstrap sample of 20 cells (D, G). (B) Receiver Operating Characteristic (ROC) curve for single cell classification. AUC=0.71. (C) Accuracy in predicting for a single PDX (cell type) its association with the category ‘Low’ versus the category ‘High’. Each data point indicates the outcome of evaluating a particular cell type by the fraction of individual cells classified as ‘Low’. N=7 PDXs: 4 low efficiency, 3 high efficiency metastasizers. 7/7 predictions are correct. Wilcoxon rank-sum and Binomial statistical test on the null hypothesis that the classifier scores of PDX with low versus high metastatic efficiency were drawn from the same distribution, p=0.0571 (Wilcoxon), p<0.00782 (Binomial). (D) Bootstrap distribution of the prediction of a PDX as a member of the ‘Low’ category. For each PDX, we generated 1000 observations by repeatedly selecting 20 random cells and recorded the fraction of these cells that were classified as ‘Low’. Horizontal line—median. Wilcoxon rank-sum test p<0.0001 rejecting the null hypothesis that the classifiers scores of observations from the two categories stem from the same distribution. This analysis demonstrated the ability to predict metastatic efficiency from samples of twenty random cells. (E-G) Discrimination results using classifiers that were blind to the cell type and day of imaging (FIG. S4A, more observations, smaller n—number of cells for each observation). (E) Receiver Operating Characteristic (ROC) curve; AUC=0.723. (F) Accuracy in predicting for one PDX on a particular day (cell type) its association with the category ‘Low’ versus the category ‘High’. Each data point indicates the outcome of testing one PDX on a particular day by the fraction of individual cells classified as ‘Low’. N=49 cell types and days: 25 low metastatic efficiency, 24 high metastatic efficiency. 32/49 predictions were correct. Wilcoxon rank-sum and Binomial statistical test on the null hypothesis that the classifier scores of PDX with low versus high metastatic efficiency are drawn from the same distribution p=0.0042 (Wilcoxon), p<0.0222 (Binomial). (G) Bootstrap distribution of the prediction of a PDX imaged in one day as member of the ‘Low’ category. See panel D. Horizontal line—median. Wilcoxon rank-sum test p<0.0001 rejecting the null hypothesis that the classifiers scores of observations from the two categories stem from the same distribution. (H) Robustness of classifier against image blur. Blur was simulated by filtering the raw images with Gaussian kernels of increased size. The PDX m528 was used to compute AUC changes as a function of blur. Representative blurred image (middle) and its reconstruction (bottom). (I) Robustness of classifier to illumination changes. AUC as a function of altered illumination (top). Representative image of m528 cell after simulated illumination alteration (middle), and its reconstruction (bottom).

FIG. 4 depicts metastatic efficiency is encoded by a single component of the latent space cell descriptor. (A) Gallery of snapshots of cells from a PDX (m610) ordered by their corresponding classifier score. (B) Approach: Each feature in the latent space cell descriptor is correlated with the score of the classifier trained to distinguish PDXs with high versus low metastatic efficiency. (C) Correlation between all 56 features (y-axis) and classifier scores for 7 PDXs (x-axis). (D) Value of feature #56 and classifier scores for individual cells color-grouped by PDX. (E) Distribution of the correlations from panel B; feature #56 (red arrow) is an obvious outlier. Left: distribution. Right: plot of log frequency for better visualization of feature #56. (F) Normalized correlation values (Z-scores) all 56 features (y-axis) and classifier scores (x-axis). Z-scores are calculated using the mean value and standard deviation of the distribution of correlation values in panel D. (G) Distribution of feature #56 values for cells grouped by association with a PDX. (H) Distribution of feature #56 values for cells grouped by association with low and high metastatic efficiency. (I) Gallery of snapshots of cells from PDX m610 in ascending order of the normalized value of feature #56. Note, high metastatic efficiency relates to negative, low metastatic efficiency to positive values of feature #56.

FIG. 5 depicts generative modeling of cell images to interpret the meaning of feature #56. (A) Approach: alter feature #56 while fixing all other features in the latent space cell descriptor to identify interpretable cell image properties encoded by feature #56. (B) Shifts in feature #56 (y-axis, measured in z-score) negatively correlated with variation in the classifier scores. (C) In silico cells generated by decoding the latent cell descriptor of a representative m498 PDX cell under gradual shifts in feature #56 (“Recon.”). Visualization of the intensity differences between consecutive virtual cells (Izscore−Izscore+0.5), only positive difference values are shown (“Diff+”). Changes in feature #56 are indicated in units of the z-score. The corresponding classifier's score and value of feature #56 are shown. (D) Approach: correlating temporal fluctuations of each feature to fluctuations in the classifiers' score. (E) Summary of correlations. Y-axis-different classifiers for each PDX. X-axis-features. Bin (x,y) records the Pearson correlation coefficients between temporal fluctuations in feature #x and the score of classifier #y over all cells of the PDX. (F) Normalization of correlation coefficients as a Z-score. Mean value and standard deviation are derived from the correlation values in panel E. (G) Following a m610 PDX cell spontaneously switching from the low to the high metastatic efficiency domain (as predicted by the classifier). Live imaging for 10 minutes. Left (top-to-bottom): raw cell image, diff+ images, classifier's score, feature #56 values. Right: visualization of the classifier score as a function of time, switching from “low” to “high” in less than 10 minutes.

FIG. 6 depicts PDX-trained classifiers predict the potential for spontaneous metastasis of mouse xenografts from melanoma cell lines. (A) All 7 PDX-trained classifiers consistently predicted that among the 6 analyzed cell lines A375 has the highest and MV3 the lowest metastatic efficiency. (B) The distribution of single cell values of feature #56 is lower for A375 than the distribution of values for MV3 cells. (C, E) Bioluminescence (BLI) of NSG mouse sacrificed 24-35 days after subcutaneous transplantation of 100 Luciferase-GFP+cells from the A375 melanoma cell line (C) versus from the MV3 cell line (E). (D, F) Bioluminescence of organs dissected from the A375 xenografted mouse (D) and from the MV3-xenografted mouse (F). 1, Gastrointestinal Tract (GI); 2, Lungs and Heart; 3, Pancreas and Spleen; 4, Liver; 5, Kidneys and Adrenal glands. In the MV3, mouse metastases were mostly found in the lungs. Black shades are mats on which the organs and mice are imaged. (G) Summary of metastatic efficiency for A375 and MV3 melanoma cell lines in 5 mice. “BLI Lungs”: Detection of BLI in the lungs. “BLI other organs”: BLI in multiple organs beyond the lungs. “Remote macro metastasis”: Macrometastases in remote organs (excluding lungs), identification of “visceral metastasis”, macrometastases visually identifiable without BLI. (H) Primary tumors in MV3 xenografts grow faster than in A375 xenografts. Mice were sacrificed 24 days after injection with MV3, 35 days after injection with A375 cells. N=5 mice for A375 and MV3 cell line. Statistics for tumor size after 24 days p-value=0.0079 (Wilcoxon rank-sum test), fold=1.6241.

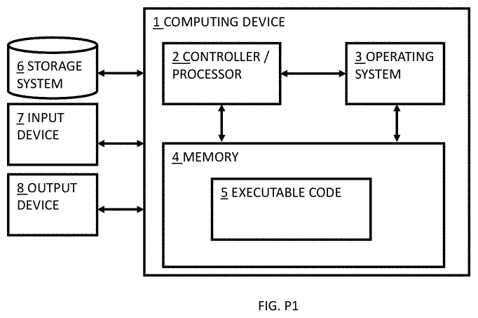

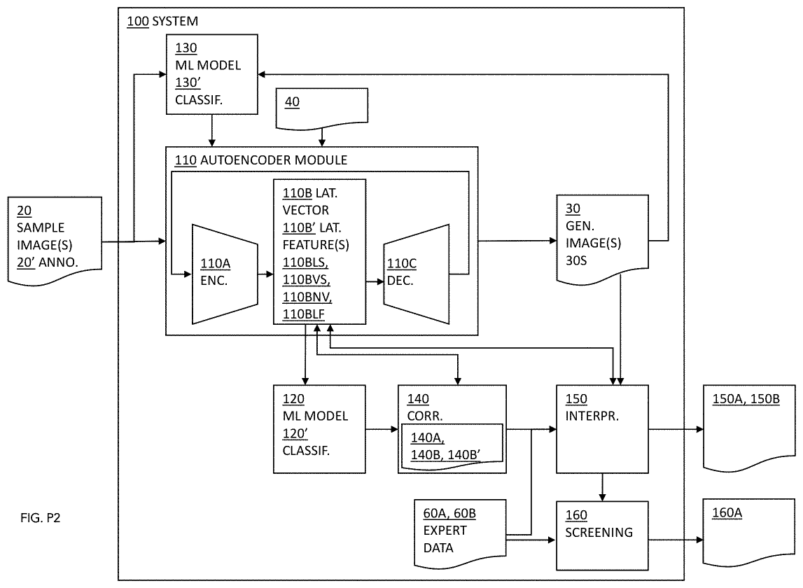

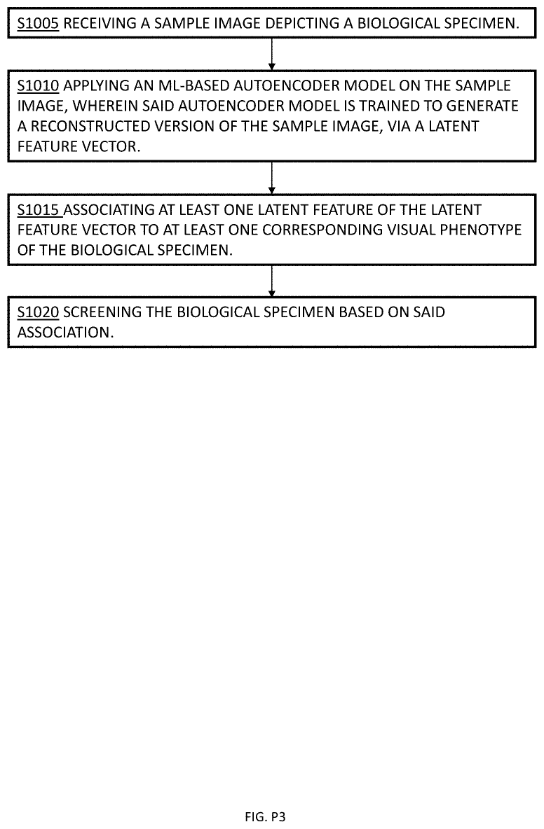

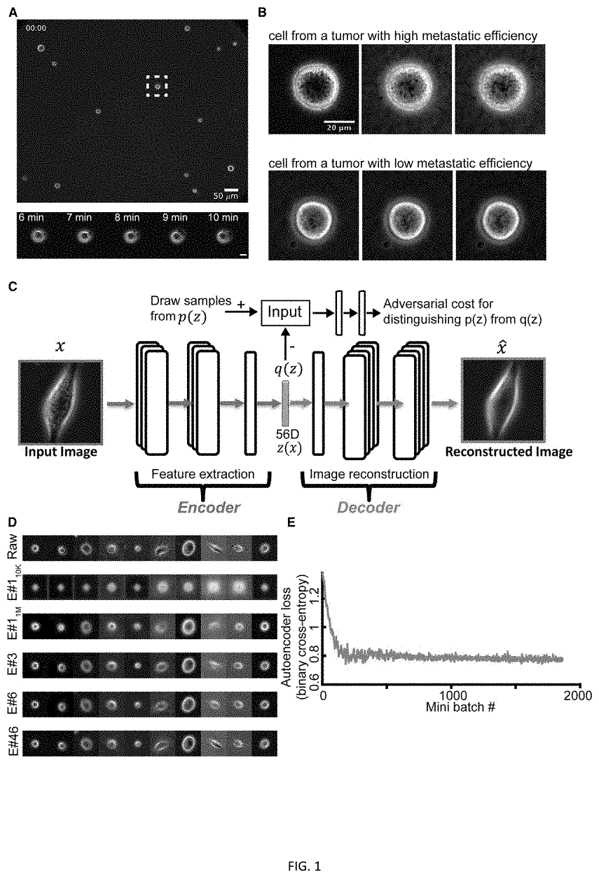

FIG. S1 depicts motility, cell shape and latent space dimensionality, Related to FIG. 1. (A) PDX melanoma on collagen were not migratory. Full field of view in phase contrast (left). Corresponding trajectories from 120 minutes indicate that cells are minimally motile (right). Only cells within the 70 μm (dashed lines) were tracked. (B) Cell shape (left: area, right: eccentricity) cannot distinguish high from low metastatic efficiency. Shown distributions were calculated from all cells in call time points. (C) Loss and image reconstruction training error as a function of the latent space dimensionality. We selected the 56-dimensional latent vector based on minimizing loss and reconstruction error. Left: autoencoder loss (binary cross-entropy) after training, right: mean square error for image reconstruction after training.

FIG. S2 depicts validation of adversarial autoencoder latent space as a quantitative measure of cell appearance. (A) Pipeline to test that increasing shifts in the latent vector of a cell relate to a monotonically increasing shift in cell appearances. (B) Increasing perturbation of a particular cell's latent space vector by Gaussian noise yields an increased deviation of the reconstructed cell image from the original image (image indicated at x=0). For each noise level, except level 0, four representative reconstructed images are shown. Lines indicate the reconstruction error for 92 randomly selected cells from different cell types and different biological replicates. (C) Cell “morphing”. Latent space interpolation shows that a gradual linear transition in latent space yields gradual transition in image space. By “gradual linear transition in latent space” we refer to constant size shifts in feature space for each shift. The trajectory goes from top-left (red) to bottom-right (green). (D) Differences of images in panel C and to the start-(red) and endpoint (green) images. (E) Cells are more self-similar over time than two neighboring cells at the same time. Two-dimensional histogram of the Euclidean distance between the latent space descriptors of a cell at time 0 and time 100 (x-axis) versus the distance of the same cell to its closest (in terms of distance in the physical space) neighboring cell in the same field of view, also at time 0 (y-axis).

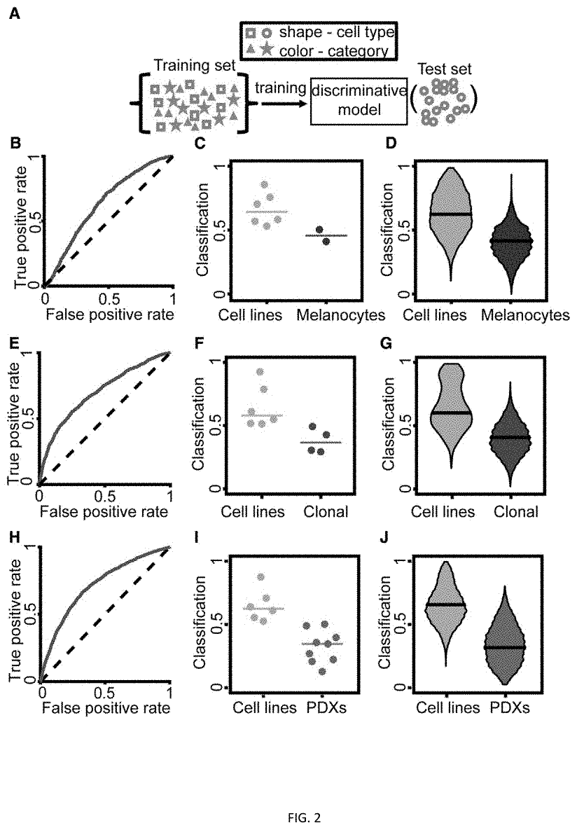

FIG. S3 depicts determining batch effects (day-to-day variability), Related to FIG. 2. Cells from four melanoma PDXs (m481, m498, m610, m634) were imaged in one batch, and this experiment was repeated on 6 different days. (A) Assessing the distance among different days for the same PDX versus the distance among the different PDXs imaged on the same day. (B) Intra-PDX/inter-day distance (x-axis) versus intra-day/inter-PDX distance (y-axis). Each dot represents the distance between the mean time-averaged latent cell descriptors averaged over all cells, arbitrary units. (C) tSNE projection of latent space cell descriptors of different PDXs on the same day (left) and of one PDX imaged on different days (right). (D) PCA projection of latent space descriptors of different PDXs on the same day (left) and of one PDX imaged on different days (right).

FIG. S4: depicts discrimination results using classifiers that were blind to the cell type and day of imaging, related to FIG. 2. (A) Blinding the cell type and the day of imaging. Multiple rounds of training and testing were performed. In each round, all cells of one cell type imaged in one day were used as the test dataset. The training set consisted of the remainder of the data, excluding the cell type at test and data from the same day of imaging. Thus, the trained model was completely blind to the test set. The model classified each cell in the test set, the overall mean classification accuracy for a specific cell type and imaging day was reported. The classifier's score of every cell was recorded and accumulated for all cell type+imaging day pair for Receiver Operating Characteristic analysis. Besides excluding batch-effects by blinding the classifier to the day of imaging, this provided us with an increased number of observations (cell type, day) at the cost of a reduced number of cells per observation. (B-C) Discriminating melanoma cell lines from melanocyte lines. (B) Receiver Operating Characteristic (ROC) curve. AUC=0.635. (C) Accuracy in predicting the label ‘cell lines’ for a single cell as opposed to the label ‘melanocytes’. Each data point indicates the outcome (fraction of cells classified as ‘cell line’) of testing the cells of one melanoma cell line or melanocyte line on a particular day. N=24: 18 cell lines, 6 melanocyte lines. 19/24 successfully predicted observations. Wilcoxon rank-sum test p=0.026. Binomial statistical test p<0.003. (D-E) Discriminating melanoma cell lines from clonally expanded cell lines. (D) Receiver Operating Characteristic (ROC) curve. AUC=0.65. (E) Accuracy in predicting the label ‘cell lines’ for a single cell as opposed to the label ‘clonal’. Each data point indicates the outcome of testing the cells of one melanoma cell line or clonal expansion line on a particular day. N=29: 18 cell lines, 11 clonal expanded cells. 22/29 successfully predicted observations. Wilcoxon rank-sum test p=0.0032. Binomial statistical test p<0.0041. (F-G) Discriminating melanoma cell lines versus PDXs. (F) Receiver Operating Characteristic (ROC) curve. AUC=0.686. (G) Accuracy in predicting the label ‘cell lines’ for a single cell as opposed to the label ‘PDXs’. Each data point indicates the outcome of testing the cells of one melanoma cell line or PDX on a particular day. N=75: 18 cell lines, 75 PDXs. 63/75 successful predicted observations. Wilcoxon rank-sum test p<0.0001. Binomial statistical test p<0.0001. (H) Pairwise discrimination of cell types. Discriminating two cell types from one another. Each data point indicates the AUC value for predicting the cell type label for single cells. Multiple rounds of training and testing were performed for each pairwise classification. In each round, data from one cell type imaged in one day was used as the test dataset, while the training set consisted of the remainder of the data, excluding data from the same day of imaging. Note that here the classifiers were blind to the day of imaging, but not to the cell type at test. The green dashed line is the mean AUC=0.66. The blue dashed line indicates the AUC level of a random classifier. p-value<0.0001 (Wilcoxon sign-rank test) rejecting the null hypothesis that pairs of different cell types cannot be discriminated.

FIG. S5 depicts discriminating cell lines from PDXs using cell shape and temporal information, related to FIG. 2. (A-B) Single cell segmentation in phase-contrast images by LEVER. (A) Examples of successful segmentation. The region outside the segmentation mask is colored black. (B) Examples of failed segmentations. (C) Three scenarios of incorporating temporal information in a cell descriptor applied to either cell shape-based features or latent space cell descriptors. (D) Accuracy in predicting the label ‘cell lines’ for a single cell as opposed to the label ‘PDXs’. Each data point indicates the outcome of testing the cells of one melanoma cell line or PDX on a particular day (FIG. S4A). Classifiers derived from cell shape-based features could not discriminate between the two labels, regardless of the mode of incorporating temporal information. In contrast, the latent space cell descriptors slightly improved with explicit consideration of temporal information and all classifier modes significantly outperformed shape-based classifiers (***−p-value<0.0001, nonparametric Wilcoxon sign-rank test. N=65 experiments of one cell type imaged in one day. The green line is the median. The dashed red horizontal line represents the random model.). (E) The latent cell descriptor outperforms shape features. Matrix visualization of the comparison of the different encodings. Fold (left), p-value (middle), log p-value (right, −3 corresponds to the p-value of 0.05). The average latent cell descriptor classification accuracy surpasses other cell encoding schemes. Stat—static, Avg.—average, BOW—bag of words. (F) Mean squared displacement analysis (MSD) analysis of single cell trajectories averaged over each cell type did not show discrimination between cell lines and PDXs. Maximal time lag of 60 frames (=minutes).

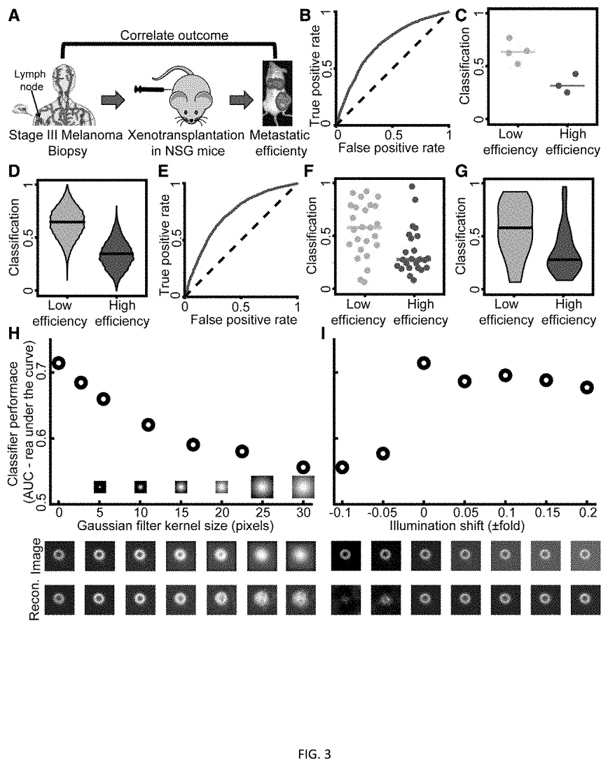

FIG. S6 depicts discriminating high versus low metastatic efficient PDXs using cell shape and temporal information, related to FIG. 3. (A) Accuracy of classifiers derived from shape based features and from latent space cell descriptors in predicting the label ‘low efficiency’ for a single cell. The classifiers include various modes of incorporating temporal information (FIG. S5). The 0.5 horizontal line reference the accuracy of a random classifier. Shape-based classifiers could not discriminate between PDXs with high and low metastatic efficiency. Classifiers derived from latent space cell descriptors performed significantly better than random, p-value<0.01 (0.0053 for autoencoder static, 0.0056 for autoencoder time average), nonparametric Wilcoxon sign-rank test. N=40 experiments of PDX imaged in one day. Green lines indicate medians of accuracy distributions. (B) Mean squared displacement (MSD) analysis of single trajectories averaged over each PDX could not distinguish between high and low metastatic efficiency. Max time lag of 60 frames (=minutes). (C) MSD analysis for a longer duration of 8 hours could not distinguish (p-value not significant in 100, 200, 300 and 480 minutes) between m498 (low metastatic efficiency, N=30 cells) versus m481 (high metastatic efficiency, N=30 cells).

FIG. S7 depicts panel of in silico cells generated by decoding a representative PDX cells' latent space cell descriptor under gradual shifts in feature #56, Related to FIG. 5. Raw images (left), reconstructed images (middle), the positive values of the intensity differences between consecutive virtual cells (right).

FIG. S8 depicts visualization of in silico cells by altering each feature highlight the unique properties of feature #56, Related to FIG. 5. Reconstructed images (left), the positive values of the intensity differences between cells with different values in feature #56 (middle), the classifiers' predicted scores (right).

FIG. S9 depicts generalized high-dimensional generative modeling for linear classifiers applied for cell lines versus PDXs discrimination. N=6 melanoma cell lines, 9 PDXs, Related to FIG. 5. (A-B) Multiple features are classification-driving for discriminating cell lines from PDXs. (A) Correlation values between all 56 features (y-axis) and the classifier scores for different cell types (x-axis). The correlation was calculated based on all cells from each cell type. (B) Normalized correlation values (Z-scores). (C) LDA coefficients trained to discriminate cell lines from PDXs (y-axis) for different cell types (x-axis). (D) LDA coefficients are correlated with feature-to-classifier-score-correlation. Each dot represents one features' correlation with the classifier (x-axis) and its corresponding LDA coefficient (y-axis). (E) Panel of in silico cells generated by decoding a representative PDX cells' latent space cell descriptor under gradual shifts along the 56-dimensional latent space weighted according to the LDA coefficients of cell lines versus PDX classification. Left-to-right describe the transition from PDX to cell line. These are the same cells used in FIG. S7. The red arrow highlights a cell where traversal of the feature space outside the natural range of data variation artificially fractionates the cell image.

FIG. S10 depicts high-dimensional generative modeling for linear classifiers applied for high versus low metastasis PDXs discrimination, related to FIG. 5. (A) Multiple features are classification-driving for discriminating high from low metastatic PDXs using latent cell descriptors derived from a second adversarial autoencoder network that was independently trained on the same dataset. (B) LDA coefficients trained to discriminate high from low metastatic PDXs (y-axis) for different PDXs (x-axis). (C) LDA coefficients are correlated with feature-to-classifier-score-correlation. Each dot represents one features' correlation with the classifier (x-axis) and its corresponding LDA coefficient (y-axis). (D) Panel of in silico cells generated by decoding a representative PDX cells' latent space cell descriptor under gradual shifts along feature #56 of the first network (left) and the 56-dimensional latent space weighted according to the LDA coefficients of the second network (right). Left-to-right describe the transition from high to low metastatic efficiency. These are the same cells used in FIG. S7.

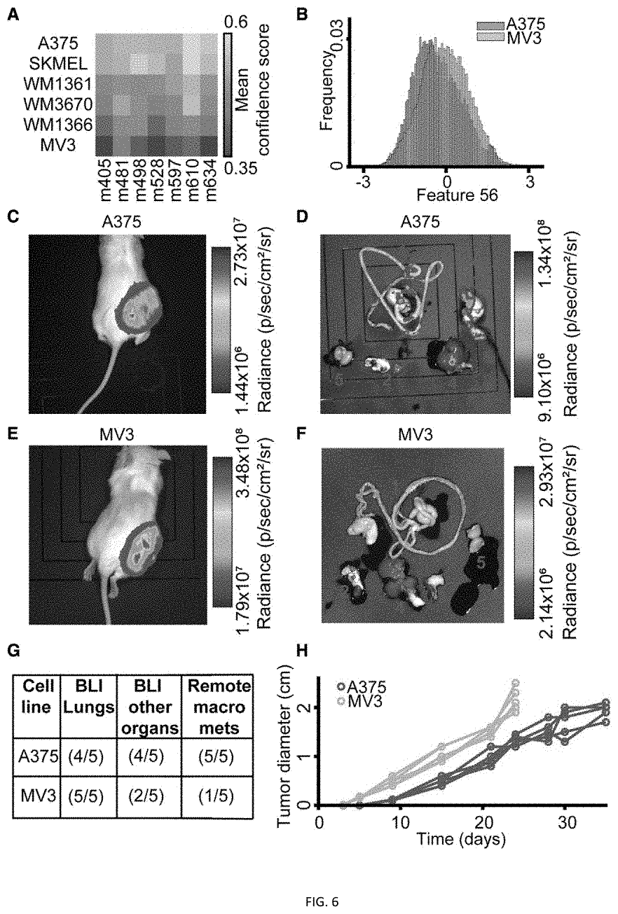

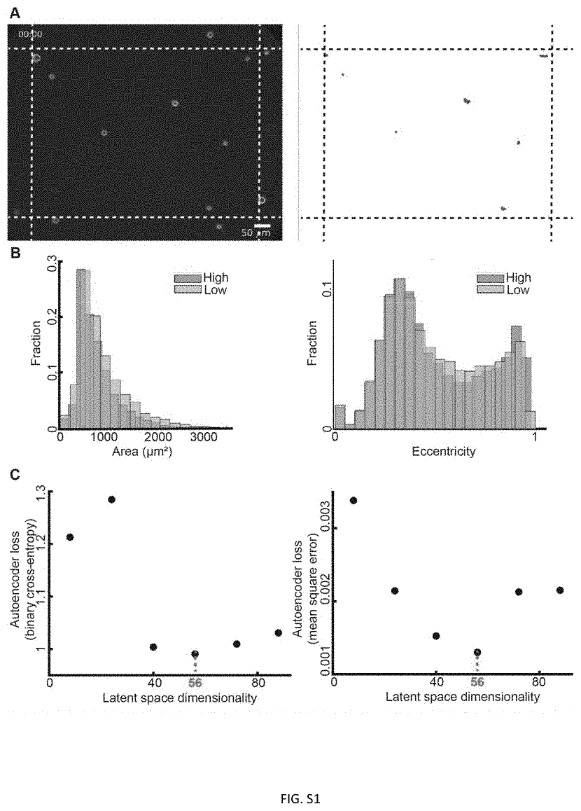

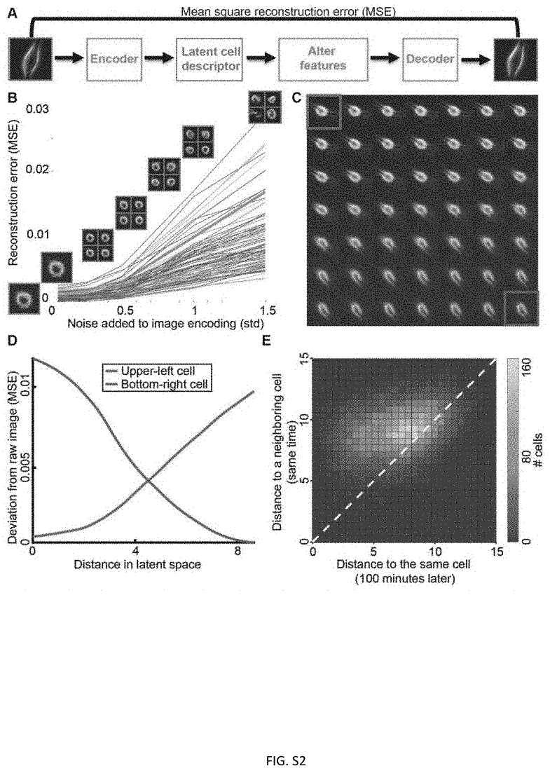

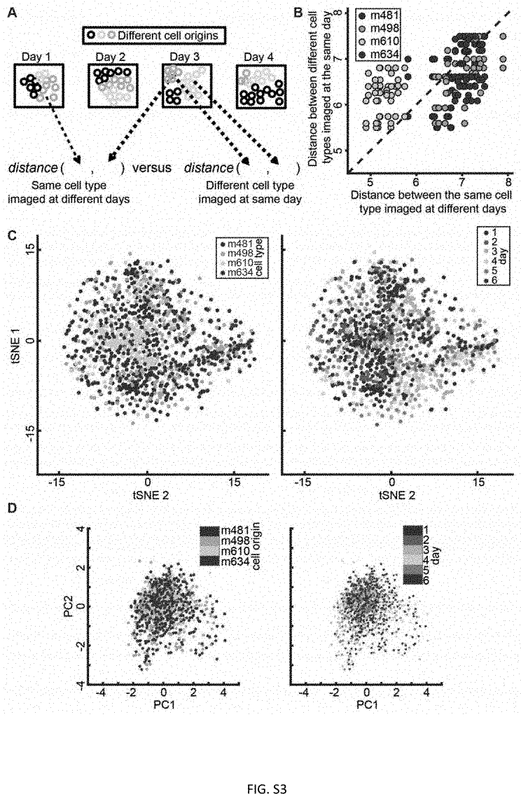

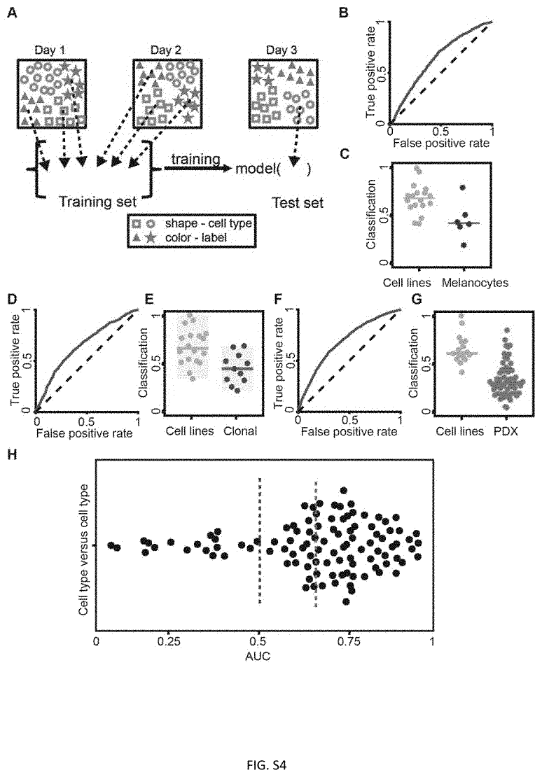

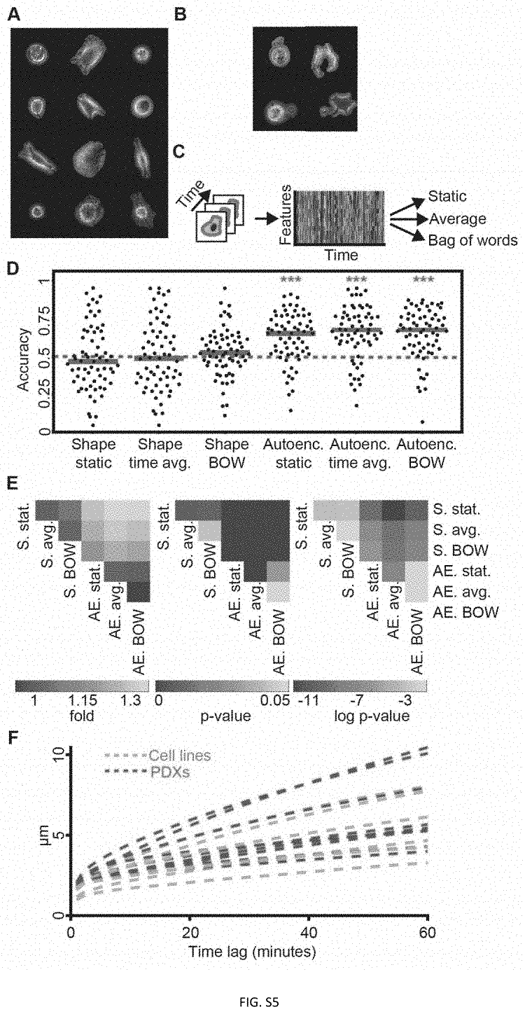

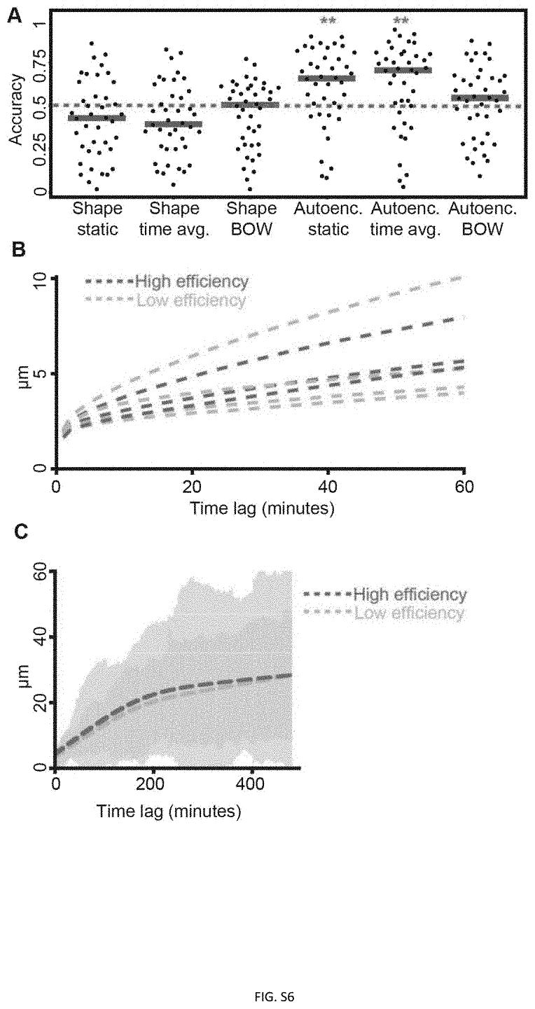

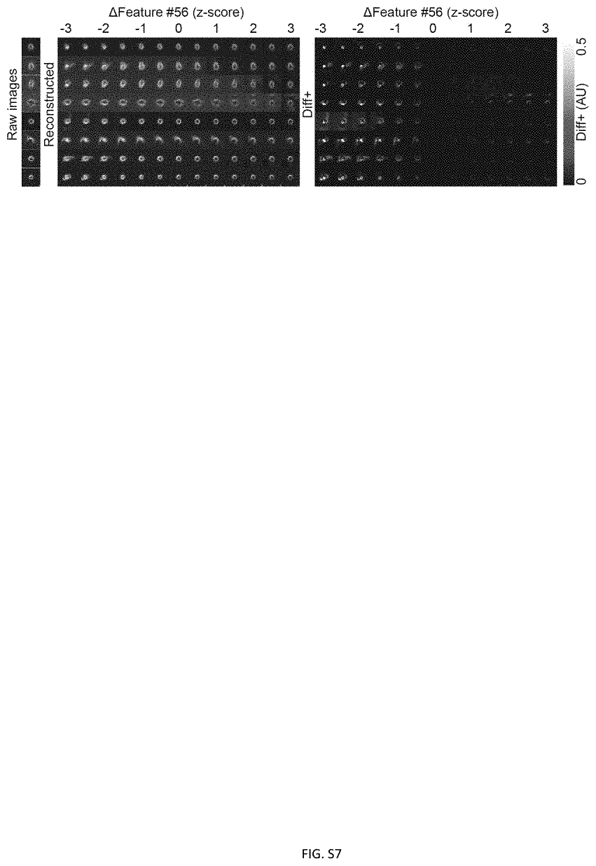

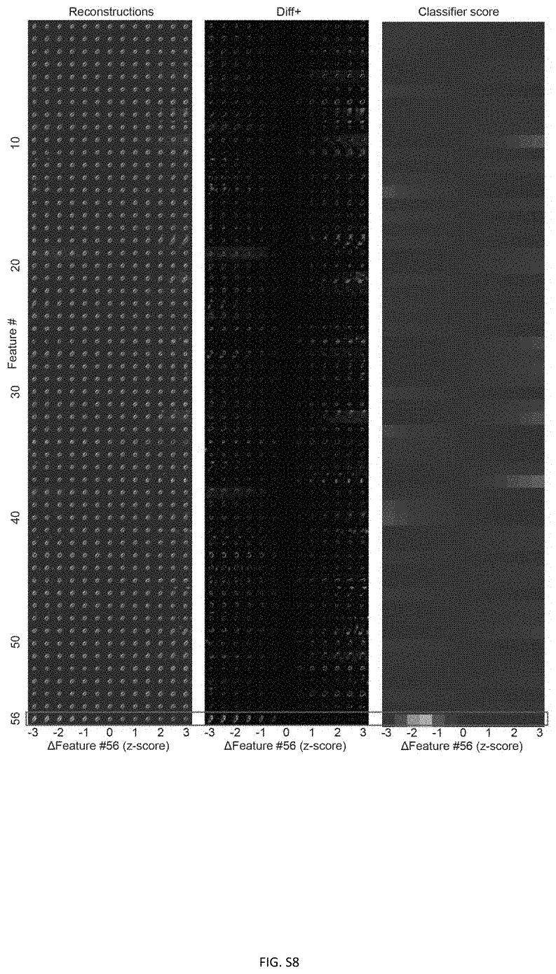

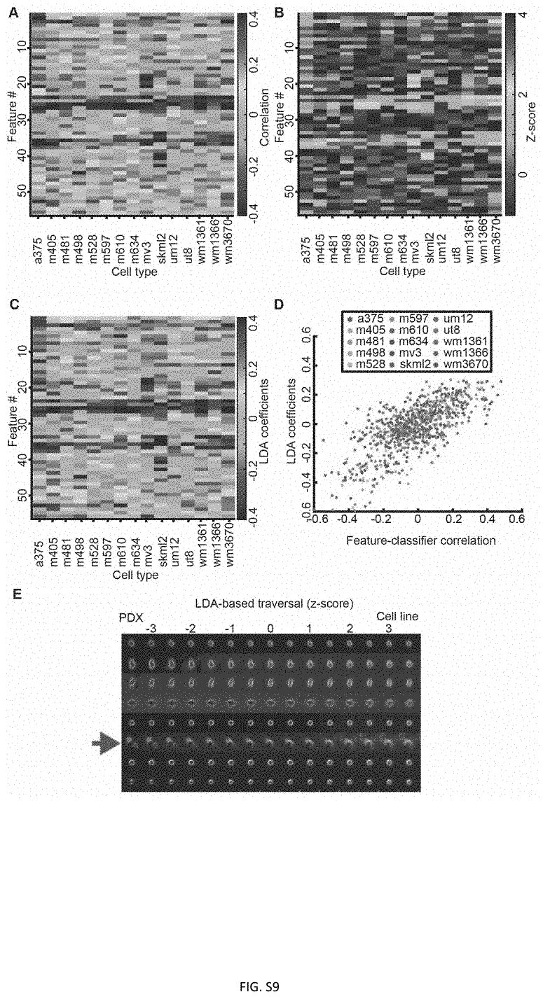

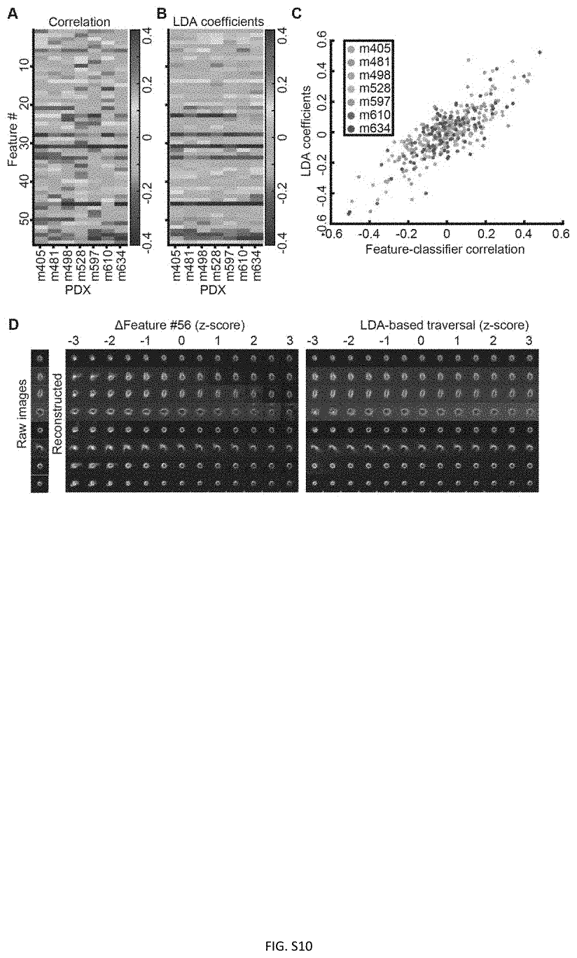

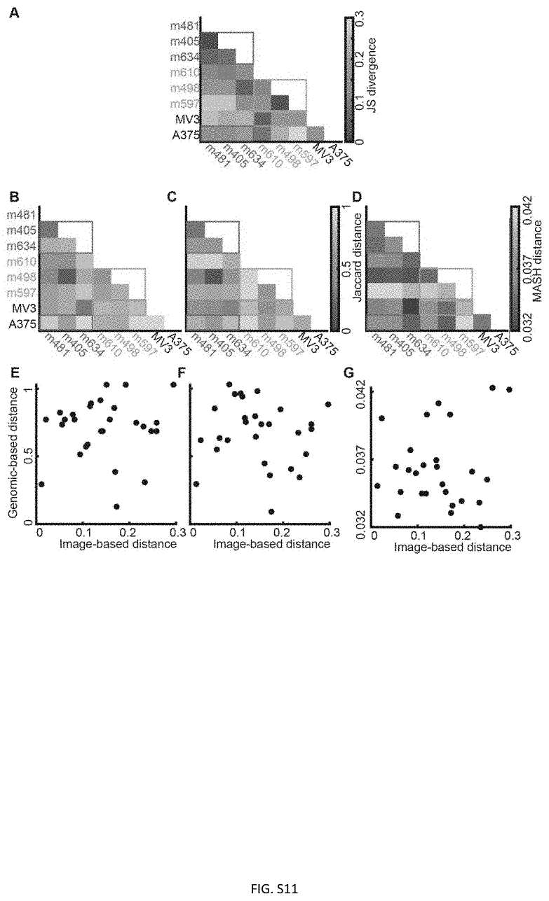

FIG. S11 depicts genomic markers could not distinguish between high and low metastatic efficiency. Distance measures between pairs of different cell types were compared between image-based (classifier scores) and genomic-mutational based information, Related to FIG. 6. (A-D) Distance matrices between pairs of cell-types. Red sub-matrices indicate the distances between PDXs (and the A375 cell line) classified as highly metastatic. Orange sub-matrices indicate the distances between PDXs (and the MV3 cell line) classified as highly metastatic. (A) Distance matric derived from image based classifier scores. Individual distances were computed based on the Jensen-Shannon divergence of the classifier score distributions for single cells in each of the compared cell types. The sub-matrices of cell types with similar levels of metastatic efficiency show low distances compared to matrix bins comparing cell types with differing metastatic efficiency. (B-D) Distance matrices derived from the genomic profiles of cell types cannot distinguish between high and low metastatic efficiency. (B) Distances calculated based on the Jaccard index of the mutational state of the oncogenic mutations in the 20 top mutated genes in melanoma. (C) Distances calculated based on the Jaccard index of the non-oncogenic mutations from those same genes. (D) Distances calculated by application of the alignment-free method MASH to the sequences from the entire 1400 gene panel. (E-G) Distances derived from image-based versus genomics-based cell-type to cell-type distinction are not correlated. Each datum holds the matched pair classifier- and genomic- distances between two cell types. E, F, and G correspond to the matrices in B, C and D, each correlating with the distance matrix in A. No correlation was found to be statistically significant.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Reference is now made to FIG. P1, which is a block diagram depicting a computing device, which may be included within an embodiment of a system for screening biological specimens, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may screen biological specimens as further described herein. Although, for the sake of clarity, a single item of executable code 5 is shown in FIG. P1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to biological specimens may be stored in storage system 6 and may be loaded from storage system 6 into memory 4 where it may be processed by processor or controller 2. In some embodiments, some of the components shown in FIG. P1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

Reference is now made to FIG. P2, which is a block diagram depicting a system 100 for screening biological specimens, according to some embodiments of the invention. System 100 may be implemented as a software module, a hardware module, or any combination thereof. For example, system may be or may include a computing device such as element 1 of FIG. P1, and may be adapted to execute one or more modules of executable code (e.g., element 5 of FIG. P1) to screen biological specimens, as further described herein.

As shown in FIG. P2, arrows may represent flow of one or more data elements to and from system 100 and/or among modules or elements of system 100. Some arrows have been omitted in FIG. P2 for the purpose of clarity.

As shown in FIG. P2, system 100 may employ a generative neural network, denoted herein as autoencoder module 110 (or autoencoder, or autoencoder model 110), in combination with one or more supervised machine learning ML classifiers, denoted herein as ML model 120 or classification module 120, to screen biological specimens as elaborated herein.

According to some embodiments, system 100 may receive a representation of at least one biological specimen, denoted sample image(s) 20. For example, sample image(s) 20 may include at least one image depicting a cell. As elaborated herein, system 100 may supervised ML classifier model 120 to classify the biological specimen (e.g., the depicted cell) according to a predefined classification.

The terms “biological specimen”, “medical specimens”, or “biomedical specimen” may be used herein interchangeably to indicate any type of information depicting, or representing biological, medical, or biomedical information.

A non-limiting example of such classification by ML model 120, which is extensively discussed herein, includes classification of biological specimen such as a cancerous cell (e.g., a melanoma cell) depicted in sample image 20, according to propensity or effectiveness of metastasis.

Another example of classification of a biological specimen depicted in sample image 20 may include prediction of a success rate of a blastocyst, to be transferred or implanted during an In-Vitro Fertilization (IVF) procedure.

Another example of classification of a biological specimen may include prediction of a probability of a neuron depicted in sample image 20, to fire a neural pulse.

Another example of classification of biological specimens may include classification of Tuberculosis Granulomas microscopy imaging 20.

Another example of classification of biological specimens may include classification of medical conditions from sample images 20 originating from a variety of imaging modalities, such as Computed Tomography (CT) scans, Magnetic Resonance Imaging (MRI) scans, pathological slides, histological images, and the like. Other applications of biological specimen classifications may also be possible.

As known in the art, autoencoder model 110 may include an encoder submodule 110A, and a decoder submodule 110C. Encoder submodule 110A may be adapted to produce a representation of input instances of data (e.g., sample images 20) in a latent vector 110B. Latent vector 110B may represent a latent space 110BLS having reduced dimensions in relation to the input data (e.g., sample images 20). Each entry of latent vector 110B may be referred herein as a latent feature 110B′. Decoder submodule 110A may be adapted to produce or generate, from latent vector 110B a reconstructed version of the input instances of data.

According to some embodiments, autoencoder model 110 may be trained to generate (e.g., via encoder 110A, intermediate latent feature vector 110B, and decoder 110C) a reconstructed version of sample image 20. This reconstructed version is denoted herein as generated image(s) 30.

As elaborated herein, in a subsequent inference stage, system 100 may apply trained ML based autoencoder model 110 on at least one instance of input sample image 20, to generate one or more corresponding generated images 30 via intermediate latent feature vector 110B.

According to some embodiments, ML model 120 may be trained to classify biological specimens depicted in sample image 20, according to one or more classes or categories, based on latent feature vector 110B. In other words, ML model 120 may be trained to produce a classification, or classification score 120′ representing pertinence of a relevant latent feature vector 110B to a predefined class. The terms classification and classification scores may be used herein interchangeably.

For example, during a training stage, system 100 may receive a plurality of expert-annotated sample images 20, where each annotated sample image 20 may include annotations or labels 20′ representing a class of a depicted biological specimen.

It may be appreciated that the unsupervised autoencoder model 110 may be trained independently of the supervised ML models 120, and does not require annotated data for the purpose of training. Therefore, system 100 may utilize unlabeled data to improve classification of biological specimens.

Pertaining to the example of depicted blastocysts, an expert annotation 20′ may include a numerical or binary value representing successful or failed implantation of the blastocyst during an In Vitro Fertilization (IVF) procedure. Pertaining to the example of depicted melanoma cells, an expert annotation 20′ may include a numerical value representing the propensity or effectiveness of metastasis.

During a training stage, system 100 may use a supervised learning, or supervised training algorithm as known in the art, to train ML model 120 to classify (e.g., produce a classification 120′ of) the depicted biological specimens of image(s) 20, based on the input of latent feature vector 110B, and supervisory data of annotations 20′.

Pertaining to the example of depicted blastocysts, during a training stage, system 100 may apply a supervised training algorithm to train ML model 120 to classify (e.g., produce a classification 120′ of) at least one blastocyst, according to implantation success, using values of features 110B′ of latent feature vector 110B as input.

Pertaining to the example of depicted melanoma cells, during a training stage, system 100 may apply a supervised training algorithm to train ML model 120 to classify (e.g., produce a classification 120′ of) at least one melanoma cell, according to the propensity of metastasis, using values of features 110B′ of latent feature vector 110B as input.

During a subsequent inference stage, system 100 may apply ML-based classification model 120 on one or more latent feature vectors 110B, to calculate corresponding classification scores 120′ representing pertinence of a relevant latent feature vector 110B to a predefined class (e.g., propensity of metastasis).

According to some embodiments, system 100 may include a correlation module 140, adapted to analyze classification 120′ of one or more biological samples in view of latent features 110B′. As elaborated herein, correlation module 140 may be configured to identify one or more (e.g., a first subset 140A) latent features 110B′ of the generative autoencoder 110 that drive, or are significantly correlated to the classification 120′ of the biological specimens by the supervised classification ML model 120. The first subset of latent features 110B′ is denoted herein as features subset 140A.

As elaborated herein, correlation module 140 may compute, for at least one latent feature 110B′ a significance score 140B, based on the classification scores 120′. Significance score 140B may represent significance of the at least one latent feature in driving an outcome or classification 120′ of ML classification model 120. Correlation module 140 may subsequently attribute or associate the calculated significance score to the relevant at least one latent feature, and select the first subset 140A of latent features 110B′ based on their respective significance scores 140B (e.g., by selecting the top-scoring latent features 110B′).

In some embodiments, significance score 140B may be calculated as a numeric value, e.g., in the range of [0,1], where high values (e.g., 0.9) represent significance of a latent feature 110B′ in driving classification score 120′, and small values (e.g., 0.1) representing insignificant or null effect of a relevant latent feature 110B′ in driving classification score 120′.

For example, correlation module 140 may be configured to collaborate with autoencoder module 110, to individually modify a value of a specific latent feature 110B′. Correlation module 140 may monitor classification score 120′ which may change following the modification of the latent feature 110B′ value. Correlation module 140 may thus calculate significance score 140B to represent a dependency (e.g., a linear regression or non-linear relation along the gradient of the classification score in the latent space) between classification score 120′ and the relevant latent feature 110B′.

Additionally, or alternatively, correlation module 140 may calculate significance score 140B to represent a correlation metric between classification score 120′ and the relevant latent feature 110B′.

Pertaining to the example of prediction of IVF success rate from an image of a blastocyst: ML model 120 may be configured to produce a classification score 120′ that includes a confidence score or confidence level, representing confidence of a depicted blastocyst to be implanted successfully during IVF. For one or more depicted blastocysts, correlation module 140 may calculate significance score 140B as a value of correlation 140B′ between one or more (e.g., each) latent feature 110B′ and the relevant blastocyst's confidence level of successful implantation. Correlation module 140 may then select one or more (e.g., a subset 140A) of latent feature 110B′ that correspond to the most extreme (e.g., most positive, or most negative) significance scores 140B (e.g., correlation values 140B′), to identify at least one latent feature 110B′ of the generative autoencoder NN 110 that significantly affects or drives the classification or prediction 120′ of successful implantation of the blastocyst.

Additionally, or alternatively, system 100 may include an interpretability module 150. As elaborated herein, interpretability module 150 may be adapted to identify one or more latent features of latent features subset 140A, that are significantly correlated with at least one visual phenotype 150B of the biological specimens depicted in sample image 20.

As elaborated herein, interpretability module 150 may be configured to produce a second subset 150A of latent features 110B′ (e.g., a sub-group of subset 140A). Subset 150A may include at least one latent feature 110B′ of the latent feature vector 110B that is associated to at least one corresponding visual phenotype 150B of the depicted biological specimen.

In other words, interpretability module 150 may associate one or more latent features 110B′ (e.g., feature subset 150A) of the latent feature vector 110B with at least one visual phenotype 150B of the depicted biological specimen, based on at least one of classification score 120′ and significance score 140B. Interpretability module 150 may perform this association based on classification score 120′ in a sense that it may only relate to relevant classifications of a depicted biological specimen. Additionally, or alternatively, interpretability module 150 may perform this association based on significance score 140B, in a sense that it may only associate latent features 110B′ of subset 150A, that are selected as significantly driving classification 120′.

Pertaining to the example of prediction of IVF success rate from an image of a blastocyst, visual phenotypes 150B of the blastocysts may include, or may represent for example Inner Cell Mass (ICM) of an embryo, a size or diameter of an embryo within the blastocyst, a trophectoderm (TE) of the embryo, and the like.

Pertaining to the example of classification of melanoma metastasis propensity, visual phenotypes 150B of the depicted cell may include, or may represent for example morphological properties of the cell (e.g., roundness, appearance of pseudopodal extensions, size, and the like) and colorization properties (e.g., contrast, brightness, light scattering, and the like).

According to some embodiments, for one or more latent features 110B′, interpretability module 150 may collaborate with autoencoder module 110 to modify a value of the latent feature to produce a vector set 110BVS, that may include a plurality of latent feature vectors 110B. Vector set 110BVS may be an ordered set, in a sense that the order of member latent feature vectors 110B in the set represents a gradual modification (e.g., gradual enhancement or amplification) of the relevant latent feature 110B′. Autoencoder module 110 may subsequently apply decoder portion 110C of the autoencoder on vector set 110BVS, to produce a corresponding reconstructed image set 30S. Image set 30S may be an ordered set of generated images 30, in a sense that the order may represent evolution (e.g., a gradual change or amplification) of a visual phenotype 150B of the biological specimen in the respective reconstructed, generated images 30.

According to some embodiments, for one or more latent features 110B′, interpretability module 150 may associate the latent feature 110B′ to a specific visual phenotype 150B based on the reconstructed image set 30S. This association may be performed semi-manually or automatically, as elaborated herein.

For example, interpretability module 150 may employ an automated image analysis algorithm on one or more images 30 of reconstructed image set 30S, to identify manifestation of visual phenotype 150B in images 30. Additionally, or alternatively, interpretability module 150 may use the automated image analysis algorithm to determine an evolution (e.g., amplification or weakening) of visual phenotype 150B in images 30 of reconstructed image set 30S, corresponding to gradual modification of a specific latent feature 110B′ value. Interpretability module 150 may then associate latent feature 110B′ to the identified visual phenotype 150B, based on the determined evolution. In other words, visual phenotype 150B of the biological specimen may be undetectable by a human observer in sample image 20, and evolution of the visual phenotype may include amplification of the visual phenotype, such that the visual phenotype is detectable by the human observer in at least one reconstructed image 30 of the reconstructed image set 30S. Pertaining to the example of a melanoma cell, visual phenotype 150B may include appearance of pseudopodal extensions, which may not be detectable by the human observer in sample image 20, but may be heightened or extended so as be detectable by the human observer in a reconstructed image 30.

In another example, interpretability module 150 may present (e.g., via a UI such as output device 8 of FIG. P1) one or more images 30 of reconstructed image set 30S to a human expert. Interpretability module 150 may prompt the human expert to provide a label data element 60B, that may mark or note a specific visual phenotype 150B that may be visible or detectable to the human expert in images 30 of image set 30S. For example, interpretability module 150 may prompt a physician to look at reconstructed images 30S, and provide a label data element 60B that identifies changes in a visual phenotype 150B, as a result of modification of a respective latent feature 110B′. Interpretability module 150 may thus associate latent feature 110B′ to the identified visual phenotype 150B, according to label data element 60B.

Additionally, or alternatively, system 100 may apply supervised ML based classification model 120 on one or more (e.g., all) latent feature vectors 110B of vector set 110BVS, to calculate corresponding classification scores 120′ representing pertinence of a relevant latent feature vector 110B to a predefined class (e.g., propensity of metastasis).

In such embodiments, interpretability module 150 may associate at least one latent feature 110B′ to visual phenotype 150B, further based on the one or more classification scores 120′.

For example, interpretability module 150 may associate latent feature 110B′ to visual phenotype 150B either automatically, or semi-manually, as elaborated above, conditioned upon a predefined value of classification 120′. In other words, interpretability module 150 may associate latent feature 110B′ to visual phenotype 150B as long as the biological specimen depicted in reconstructed image 30 (as represented by modified latent feature vector 110B) corresponds to a relevant class.

Pertaining to the example of classification of melanoma metastasis propensity, interpretability module 150 may associate latent feature 110B′ to visual phenotype 150B when (e.g., only when) classification 120′ indicates that modified latent feature vector 110B is classified as pertaining to the class of high propensity of metastasis.

It may be appreciated that by correlating or associating latent features 150A of the generative autoencoder NN 110 with visual phenotypes 150B of the depicted biological specimens, system 100 may provide much needed interpretability or understanding of latent features 110B′ upon which classifications 120′ are based.

As elaborated herein, visual phenotypes 150B, may be associated by interpretability module 150 to specific latent features 150A, which in turn have been selected as significant for classification-driving features by correlation module 140. It may thus be appreciated that visual phenotypes 150B may represent underlying root causes, or sub-classifications of a relevant classification problem.

Pertaining to the example of prediction of IVF success rate from an image of a blastocyst, system 100 may provide, or highlight to an expert a manifestation of visual phenotypes 150B (e.g., size, ICM, TE) that may be a root cause for classification of a blastocysts as either successful or failed to implant.

Pertaining to the example of melanoma cell metastasis propensity, system 100 may provide, or highlight to an expert a manifestation of visual phenotypes 150B (e.g., appearance of pseudopodal extensions or alteration of light scattering due to change in the cell's organelle composition) that may be a root cause for the classification of a cell as being highly metastatic or not.

It has been experimentally observed that visual phenotypes 150B may be initially too subtle to be noticed or identified by an expert human viewer (e.g., a physician). According to some embodiments, autoencoder module 110 may be adapted to receive (e.g., via a user interface (UI) such as input device 7 of FIG. P1) at least one request 40 for amplifying, or exaggerating a value of one or more visual phenotypes 150B (e.g., enhance appearance of pseudopodal extensions). Autoencoder module 110 may collaborate with interpretability module 150 to identify at least one latent feature 110B′, that corresponds to, or is associated with the one or more visual phenotypes 150B of request 40. Autoencoder module 110 may thus modify (e.g., increase or decrease) a value of the identified at least one latent feature 110B′, resulting in an amplified manifestation of the requested visual phenotypes 150B in generated image 30.

It may be appreciated that by identifying specific latent features 110B′ that (a) drive classification 120′ (e.g., included in subset 140A) and (b) are interpreted as associated with specific visual phenotypes 150B (e.g., included in subset 150B), system 100 may enable a user to screen the depicted biological specimens.

For example, generated images 30 may be presented on a user interface (UI) such as output device 8 of FIG. P1, allowing an expert user to perform separate, gradual modification of individual screening operations. In other words, system 100 may allow an expert user to perform semi-manual screening operations 160A of the depicted biological specimens, in relation to specific screening operations. System 100 may then provide screening operations 160A, or an indication or notification of screening operations 160A, e.g., via a UI (such as input device 7 and/or output device 8 of FIG. P1).

Screening operations 160A may include, for example diagnostic evaluation of underlying causes (e.g., relating to appearance of visual phenotypes 150B of the biological specimen), that contribute to a specific classification 120′ of interest (e.g., identification of a cell as metastatic). For example, system 100 may be communicatively connected to a diagnostic database (e.g., element 6 of FIG. P2), that may include a table of diagnoses of sub types of classification 120′, each related to one or more specific visual phenotypes 150B. In the example of melanoma cells, such diagnostic sub types may include sub-classifications of a melanoma cell, e.g., as a melanoma cell with pseudopodal extensions and/or a melanoma cell with increased light scattering, as elaborated herein. System 100 may collaborate with diagnostic database 6, and produce a notification of the suspected diagnosis via a UI.

In another example, screening operations 160A may include performing triage of a biological specimen based on an underlying, subtle visual phenotype 150B (e.g., rather than on an overall classification 120′ result). For example, system 100 may collaborate with diagnostic database 6 to perform triage (e.g., determine urgency of treatment, determine additional required tests, and the like), based on the subtype of classification 120′, and produce a notification of the suspected diagnosis via a UI (e.g., elements 7, 8 of FIG. P1).

In yet another example, screening operations 160A may include applying, or providing a recommendation to apply a treatment agent (e.g., prescribe a drug, determine a dosage of a drug, recommend radiation treatment, recommend chemotherapeutic treatment, and the like) based on the subtle visual phenotype 150B (e.g., based on the subtype of classification 120′).

Additionally, or alternatively, system 100 may include a screening module 160, configured to utilize the obtained interpretability, so as to automatically screen (e.g., perform screening operations 160A) on one or more biological specimens, based on visual phenotypes 150B (e.g., based on the association of visual phenotypes 150B to latent features 150A).

For example, screening module 160 may receive one or more rule data elements 60A representing definitions or rules in relation to visual phenotypes 150B of biological specimens depicted in image(s) 20. Such rule data elements 60A may, for example, be implemented as a data structure (e.g., a table) that may associate between specific values, or ranges of visual phenotypes 150B, specific classifications 120′, and corresponding screening actions 160A.

Screening module 160 may collaborate with interpretation module 150 to translate visual phenotype values 150B of rules 60A to corresponding values of latent features 110B′ included in subset 150A. Screening module 160 may further collaborate with ML classification model 120, to obtain a classification score 120′ corresponding to an input instance of a sample image 20. Screening module 160 may subsequently apply at least one screening action 160A based at least in part on the classification score 120′ and/or the translated values of latent features 110B′.

In other words, screening module 160 may highlight (e.g., to an expert, via a UI) a visual phenotype 150B (e.g., a morphological characteristics, color characteristics, etc.) that drives a specific classification 120′ (e.g., propensity of metastasis) of a depicted biological specimen. Additionally, or alternatively, screening module 160 may perform screening (e.g., perform diagnosis, apply a treatment agent, and the like) based on, or specific to an underlying subtle visual phenotype 150B, in view of an overall classification 120′ result.

As elaborated herein, autoencoder 110 may collaborate with at least one of correlation module 140 and/or interpretability module 150, to modify a value of at least one latent feature 110B′, thus generating at least one modified latent feature vector 110B. As elaborated herein, system 100 may apply a variety of algorithms on autoencoder 110, to modify latent feature 110B′.

For example, for one or more latent features 110B′, system 100 may determine a range and/or a step size, so as to define a latent feature space of the autoencoder model 110, and modify the value of the one or more latent features 110B′ so as to traverse through the latent space 110BLS of the autoencoder model 110, e.g., by sampling the one or more latent features 110B′ within the determined range, and/or according to the determined step size.

Additionally, or alternatively, for at least one latent feature 110B′, system 100 may accumulate (e.g., in data storage 6 of FIG. P1) a plurality of original values of that latent feature 110B′, corresponding to, or originating from a plurality of original sample images 20. System 100 may calculate a natural variation data element (denoted 110BNV) of a latent feature space, according to, or as defined by the latent feature vector.

For example, system 100 may calculate a range, a mean value, and/or a standard deviation value of one or more (e.g., each) latent feature 110B′, based on the accumulated latent feature 110B′ values, and subsequently compute natural variation 110BNV as a multidimensional data structure, representing statistics (e.g., range, mean value, standard deviation, and the like) of one or more (e.g., all) member latent feature 110B′.

Autoencoder 110 may then traverse through the latent space 110BLS of the autoencoder model 110, based on an “exaggeration” of normal, or original latent feature 110B′ values.

For example, system 100 may modify the value of at least one latent feature 110B′ in an “exaggerated” manner, beyond the calculated natural variation of the calculated latent feature space 110BNV. Modified latent feature 110B′ may be regarded as exaggerated in a sense that it may be set beyond the calculated range of 110BNV, beyond a predefined number of calculated standard deviations from a calculated mean value of 110BNV, and the like.

As elaborated herein, visual phenotypes (e.g., altered light scattering) 150B, which may represent an underlying cause for classification 120′ of a biological specimen (e.g., having high propensity of metastasis) are typically too subtle to be noticed or identified by an expert human viewer.

It may therefore be appreciated that (a) the selection of specific latent features 110B′ that significantly (e.g., having high significance score 140B) drive classification 120′ and (b) exaggeration of these specific latent features 110B′ may allow system 100 to identify weak visual signals in image 20 as pertinent to classification 120′.

In other words, the above steps (a) and (b) may allow system 100 to generate or identify visual phenotypes that lie far beyond a range of experimental data. Such “exaggeration” of latent features 110B′ (and subsequently—the respective visual phenotypes 150B) may enable interpretation of latent features 110B′, in a sense of associating specific latent features 110B′ to the “exaggerated”, respective visual phenotypes 150B. As elaborated herein, interpretation module 150 may subsequently perform this association, based on the exaggerated latent features 110B′ either semi-manually, or automatically.

For example, interpretation module 150 may perform the association semi-manually, in a sense that it may present generated images 30 to an expert user, and prompt them for labels in relation to specific visual phenotypes 150B, as elaborated herein. In another example, interpretation module 150 may perform the association automatically, by employing predefined image analysis algorithms to identify key sample behaviors that permit or drive classification 120′.

Additionally, or alternatively, autoencoder module may modify the value of the one or more latent features 110B′ so as to traverse through the latent space 110BLS of the autoencoder model along a predetermined, optimal trace.

For example, system 100 may select one or more latent features 110B′ based on their attributed significance scores 140B (e.g., having top-most significance scores 140B). System 100 may then set a trace for traversing through the latent space 110BLS in an iterative, or repetitive process.

In each step or iteration of the process, system 100 may calculate a trajectory that follows a gradient of classification score 120′ based on (e.g., in the coordinates of) the selected one or more latent features 110B′. Autoencoder 110 may modify the value of the one or more selected latent features 110B′ according to the calculated trajectory, thus performing a “step” through the latent space 110BLS . System 100 may continue or repeat these iterations so as to traverse through the latent space 110BLS of the autoencoder model.

As known in the art, autoencoder model 110 may be trained so as to minimize a cost function, where the cost function represents a value of a difference metric between the original input data (e.g., sample image 20) and an output, reconstructed data (e.g., generated image 30).

For example, during a preliminary (e.g., training) stage, system 100 may apply autoencoder model 110 on one or more (e.g., a plurality of) training sample images 20 depicting biological specimens, so to produce a corresponding reconstructed versions of the one or more training sample images 20. system 100 may calculate a loss function value 110BLF, based on the one or more training sample images 20 and corresponding reconstructed images 30. System 100 may then train autoencoder model 110 (e.g., train encoder 110A and decoder 110C) so as to minimize said loss function value 110BLF.

For example, loss function value 110BLF may represent a comparison between the one or more training sample images 20 and corresponding reconstructed images 30, and system 100 may calculate loss function value 110BLF such that reconstructed images 30 may be similar to corresponding sample images 20.

Additionally, or alternatively, system 100 may include at least one ML-based classification model 130, trained to calculate a classification score 130′ that represents pertinence of a biological specimen, depicted in an image (e.g., sample image 20) to a predefined class. As elaborated herein, system 100 may calculate loss function value 110BLF further based on classification score 130′ of ML classification model 130, and train autoencoder model 110 (e.g., training of encoder 110A and decoder 110C) so as to minimize loss function value 110BLF. It may be appreciated that such training of autoencoder model 110 may prevent loss of information that may be relevant or critical for producing classification 120′.

For example, system 100 may apply classification model 130 on at least one sample image to produce a first classification score 130′ and apply classification model 130 on the reconstructed version 30 of sample image 20 to produce a second classification score 130′. System 100 may calculate loss function value 110BLF based, at least in part on the first classification score 130′ and second classification score 130′, and train autoencoder 110 (e.g., train encoder 110A and decoder 110C) based on (e.g., to minimize) calculated loss function value 110BLF.

Additionally, or alternatively, system 100 may calculate a second loss function value 110BLF, representing discrepancy between a classification score (e.g., 120′/130′) of sample image 20 and a classification score (e.g., 120′/130′) of, or originating from reconstructed image 30, and train the autoencoder model further based on (e.g., to minimize) the second loss function value.

Additionally, or alternatively, system 100 may calculate loss function value 110BLF as a weighted sum of a first value, representing a difference between image 20 and image 30, and a second value, representing a difference between classification 130′ of image 20 and classification 130′ of image 30, and train autoencoder 110 so as to minimize loss function value 110BLF.

Reference is now made to FIG. P3, which is a flow diagram depicting a method of screening biological specimens by at least one processor, according to some embodiments of the invention.

As shown in step S1005, the at least one processor (e.g., processor 2 of FIG. P1) may receive a sample image (e.g., sample image 20 of FIG. P2) depicting a biological specimen.

As shown in step S1010, the at least one processor 2 may apply an ML based autoencoder model (e.g., autoencoder 110 of FIG. P2) on sample image 20. As elaborated herein (e.g., in relation to FIG. P2) autoencoder model 110 may be trained to generate a reconstructed image 30, which is a reconstructed version 30 of sample image 20, via a latent feature vector (e.g., latent feature vector 110B of FIG. P2).

As shown in step S1015, the at least one processor 2 may employ an interpretation module (e.g., interpretation module 150 of FIG. P2), to associate at least one latent feature (e.g., latent feature 110B′ of FIG. P2) of the latent feature vector 110B to at least one corresponding visual phenotype (e.g., visual phenotype 150B of FIG. P2) of the biological specimen.

As shown in step S1020, and as elaborated herein (e.g., in relation to FIG. P2) the at least one processor 2 may subsequently screen the biological specimen based on this association.

According to some embodiments, one or more ML models 120 of system 100 may classify 120′ patient-derived melanoma xenografts as ‘efficient’ or ‘inefficient’ metastatic, validate predictions regarding melanoma cell lines with unknown metastatic efficiency in mouse xenografts, and use generative autoencoder network 110 to generate “in silico”, reconstructed cell images 30 that amplify or exaggerate critical predictive cell properties.

These exaggerated images 30 unveiled visual phenotypes 150B such as pseudopodal extensions and increased light scattering as hallmark properties of metastatic cells.

We validated this interpretation using live cells spontaneously transitioning between states indicative of low and high metastatic efficiency classifications 120′. This study illustrates how the application of Artificial Intelligence can support the identification of cellular properties that are predictive of complex phenotypes and integrated cell functions, but are too subtle to be identified in the raw imagery 20 by a human expert.

Recent machine learning studies have impressively demonstrated that label-free images contain information on the molecular organization within the cell. These studies relied on generative models that transform label-free to fluorescent images, which can indicate the organization and, in some situations, even the relative densities of molecular structures. Models were trained by using pairs of label-free and fluorescence images subject to minimizing the error between the fluorescence ground-truth image and the model-generated image. Other studies used similar concepts to enhance imaging resolution by learning a mapping from low-to-high resolution. Common to all these studies is the concept that the architecture of a deep convolutional neural network can extract from the label-free or low-resolution cell images hidden information—also referred to as latent information—that is predictive of the molecular organization of a cell or its high-resolution image, yet escapes the human eye.

We wondered whether this paradigm could be applied also to the prediction or classification 120′ of complex cell states that result from the convergence of numerous structural and/or molecular factors. We combined unsupervised generative deep neural networks such (e.g., autoencoder 110) and supervised machine learning to train a classifier (e.g., ML model(s) 120) to predict the metastatic efficiency of human melanoma cells.

The power of cell appearance for determining cell states that correlate with function has been the basis of decades of histopathology. Cell appearance has been established as an explicit predictor of signaling states that are directly implicated in the regulation of cell morphogenesis. Whether cell appearance is also informative of a broader spectrum of cell signaling programs, such as those driving processes in metastasis, is less clear, although very recent work, using conventional shape-based machine learning of fluorescently labeled cell lines, suggests this may be the case.

The paradigm of extracting latent information via deep convolutional neural networks from label-free and time-resolved image sequences holds particularly strong promise for a task of this complexity. The design of cell appearance metrics that encode the state of, e.g., a cellular signal that promotes cell survival or proliferation, exceeds human intuition. The flip side of learning information that classifies well but is non-intuitive is the discomfort of relying on a ‘black box’. Especially in a clinical setting, the lack of a straightforward meaning of key drivers of a classifier is a widely perceived weakness of deep learning systems. Here, we demonstrate a mechanism to overcome this problem: By generating “in silico” cell images that were never observed experimentally we “reverse engineered” the physical properties of the latent image information that discriminates melanoma cells with low versus high metastatic efficiency. These results demonstrate that the internal encoding of latent variables in a deep convolutional neural network can be mapped to physical entities predictive of complex cell states. More broadly, they highlight the potential of “interpreted artificial intelligence” to augment investigator-driven analysis of cell behavior with an entirely novel set of hypotheses.

Experiments of patient-derived xenotransplantation (PDX) have been performed to test whether the latent information extracted from label-free live cell movies can predict the metastatic propensity of melanoma. During these experiments, tumor samples from stage III melanoma patients were taken and repeatedly transplanted between immuno-compromised mice. All tumors grew and eventually seeded metastases in the xenograft model. Whereas some tumors seeded widespread metastases in various distant organs, referred to as a PDX with high metastatic efficiency, other tumors mainly seeded only lung metastases, referred to as a PDX with low metastatic efficiency. Low efficiency PDXs originated from patients that were cured after surgery and chemotherapeutic treatment. High efficiency PDXs originated from patients with fatal outcome.

For this study, we had access to a panel of nine PDXs, seven of which had known metastatic efficiency and matching patient outcome. For the remaining two PDXs, the metastatic efficiency, including patient outcome, was unknown. To define the genomic states of the PDXs with known metastatic efficiency, we sequenced a panel of ˜1400 clinically actionable genes and found that the PDXs span the genomic landscape of melanoma mutations, including mutations in BRAF (5/6), CKIT (2/6), NRAS (1/6), TP53 (2/6), and copy number variation (CNV) in CDKN2A (6/6) and PTEN (3/6). For one PDX (m528), we were unable to generate sufficient genomic material for sequencing, although the cell culture was sufficiently robust for single cell imaging.

Reference is also made to FIG. 1, which is a schematic diagram depicting unsupervised learning of a latent vector that encodes characteristic features of individual melanoma cells.

In order to prevent morphological homogenization and to better mimic the collagenous ECM of the dermal stroma, we imaged cells on top of a thick slab of collagen. The cells were plated sparsely to focus on cell-autonomous behaviors with minimal interference from interactions with other cells. As depicted in panel A of FIG. 1, for each plate, we recorded with a 20×/0.8NA lens phase contrast movies of at least 2 hours duration, sampled at 1 minute intervals. Each recording sampled 10-20 randomly distributed fields of view from 1-4 plates of different cell types, each containing 8-20 individual cells.

We complemented the PDX data set with equivalently acquired time-lapse sequences of two untransformed melanocyte cell lines and six melanoma cell lines. The former served as a control to test whether the latent information allows at minimum the distinction of untransformed and metastatic cells. The latter served as a control to test whether the latent information allows the distinction of different cell populations, which, by the long-term selection of passaging in the lab, likely have drifted to a spectrum of molecular and regulatory states that differs from the PDX.

In total, our combined data set comprises time-lapse image sequences of more than 12,000 single melanoma cells, resulting in approximately 1,700,000 raw images. The cells were typically not migratory but displayed variable morphology and local dynamics. Many of the cells were characterized by an overall round cell shape and dynamic surface blebbing (FIG. S1A), regardless of whether they belonged to the melanoma group with high or low metastatic efficiency (FIG. 1B, FIG. S1B), which is consistent with reports of primary melanoma behavior in vivo and on soft substrates in vitro. Thus, we speculated that cell shape or motion might not be informative of the metastatic state of a melanoma cell.

Nonetheless, we still noted textural variation and dynamics between individual cell images. Thus, we wondered whether these images contain visually unstructured signal that could predict the metastatic propensity of a cell.

After detection and tracking of single cells over time, we used the cropped single cell images as atomic units to train an adversarial autoencoder 110, and as depicted in FIG. 1C. Autoencoder 110 may include a deep convolutional neural network denoted herein as an encoder 110A, adapted to encode image data 20 depicting a single cell into a feature vector 110B of latent information. Autoencoder 110 may further include a structurally symmetric deep convolutional neural network, denoted herein as a decoder 110C, adapted to decode feature vector 110B so as to generate one or more synthetic, reconstructed images 30 (FIG. 1C).

The encoder 110A and decoder 110C networks may be trained to minimize discrepancy between input 20 and reconstructed images 30. The adversarial component may penalize randomly generated latent cell descriptors q(z) that the network fails to distinguish from latent cell descriptors drawn from the distribution of observed cells p(z), thus ensuring regularization of the latent information space.

The trained autoencoder 110 latent space 110BLS defined a faithful metric for discriminating images of cells that appear morphologically different (FIG. S2). The autoencoder 110 training was agnostic to the subsequent classification 120′ task. The goal of this step was to determine for each melanoma cell an unsupervised latent cell descriptor that holds a compressed representation of a cell image for further classification of cell states. The terms latent descriptor, latent cell descriptor and latent feature vector 110B may be used herein interchangeably.

Experimental results have shown that latent space 110BLS cell descriptors 110B seemed to be distorted by batch effects related to inconsistencies in different imaging sessions such as operator, microscope, and gel preparation (FIG. S3). These systematic but meaningless variations in the data are a major hurdle in classification tasks. To address this issue, we transformed the autoencoder latent space 110BLS into a classifier space that was robust to inter-day confounding factors, but discriminated between different cell categories. A cell category was defined as a set of multiple cell types with a common property. For example, the category “cell line” comprises six different cell types: A375, MV3, WM3670, WM1361, WM1366, and SKMEL2 The discrimination was accomplished by training supervised machine learning models 120 on the latent cell descriptor 110B using Linear Discriminant Analysis (LDA) at the single cell level. Our intuition was that the diversity of the training data, in terms of cell categories and range of batch effects, makes the LDA classifier space robust. We validated the models in multiple rounds of training and testing, each round with the imaging data of one cell type (i.e., a specific cell line or PDX) designated as the test-set, while the rest of the data was used as the training set (FIG. 2A). Hence, the discriminative model was trained with information fully independent of the cell type it was tested on.

The number of cells from each category was balanced during training to eliminate sampling bias. To overcome the limited statistical power due to the small number of cell types (two melanocytes, four clonal expansions, six cell lines and nine PDXs), we also considered test datasets defined by all cells from one cell type imaged in one day. In this case, the training dataset included the remainder of all imaging data, except cells of any type imaged on the same day or cells of the same type on any other day (FIG. S4A). These approaches were successful in discriminating transformed melanoma cell lines from non-transformed melanocyte cell lines (FIG. 2B-D, FIG. S4B-C), melanoma cell lines from clonal expansions of these cell lines (FIG. 2E-G, FIG. S4D-E), and melanoma cell lines from patient-derived xenografts (PDX) (FIG. 2H-J, FIG. S4F-G). We also found that in pairwise comparisons most cell types could be discriminated from one another (FIG. S4H). Our latent space descriptor 110B surpassed simple shape-based descriptors attained by phase contrast single cell segmentation, and it did not benefit from either explicit incorporation of temporal information or mean square displacement analysis of trajectories (, FIG. S5). Based on these findings we used the time-averaged latent space cell descriptors 110B as the basic feature set for cell classification throughout the remainder of our study.

Although the classification performance was moderate at the single cell level (e.g., AUC of cell lines versus PDXs was 0.71, FIG. 2H), each imaging session included enough cells to accurately categorize cells at the population level (e.g., 14/15 successful cell lines versus PDXs predictions at the population level, FIG. 2I). Altogether, these results established that the latent cell descriptor 110B captures information on the functional cell state that is distinct for different cell categories and types.

Equipped with the latent space cell descriptors 110B and LDA classifiers 120, we tested our ability to predict the metastatic efficiency of single cells from melanoma stage III PDXs (FIG. 3A). Our approach was able to perfectly discriminate between the categories of melanomas with high, versus low metastatic efficiency (FIGS. 3B-3D). It was also successful at distinguishing single cells from PDXs with low versus high metastatic efficiency that were imaged on a single day (small number of cells), by classifiers 120 that were blind to the PDX and to the day of imaging (FIG. S4A, FIG. 3E-3G). Cell shape information (FIG. S6A) and mean square displacement analysis of the cell trajectories in space (FIG. S6B-S6C) could not stratify PDXs along these two categories. Classifiers 120 trained with the latent space cell descriptor 110B were robust to artificial blurring (FIG. 3H), and illumination changes (FIG. 31). These results established the potential of the proposed imaging and analytical pipeline as a diagnostic, live cytometry approach.

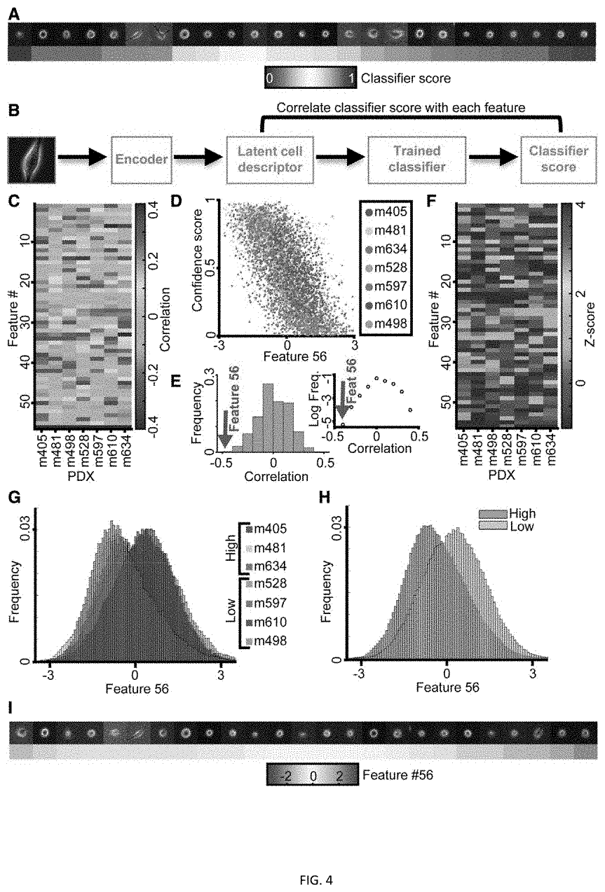

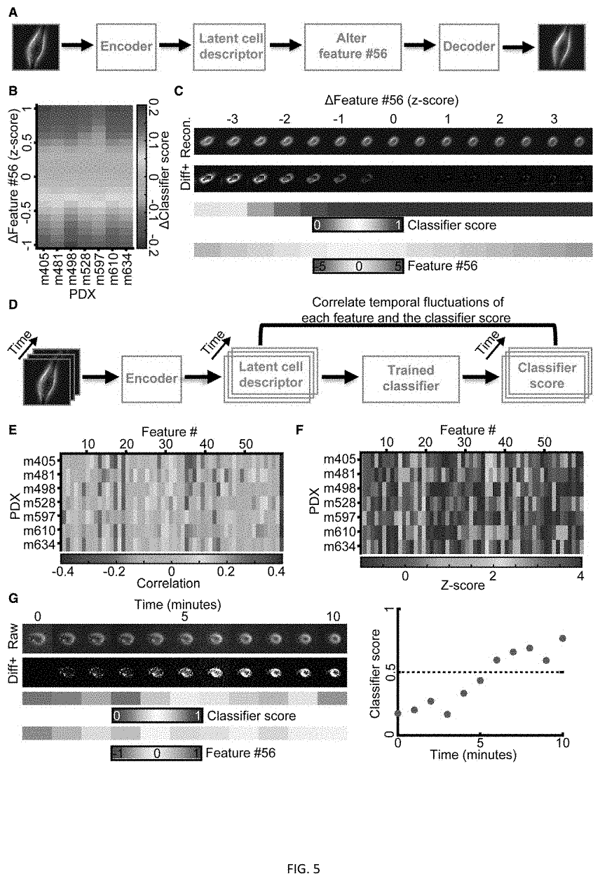

Our results thus far established the predictive power of the latent cell descriptor 110B for the diagnosis of classification 120′ of metastatic potential. However, the power of these deep networks to recognize statistically meaningful image patterns that escape the attention of a human observer is also its biggest weakness: What is the information extracted in the latent space that drives the accurate classification 120′ of low versus high metastatic PDXs? When we plotted a series of cell snapshots from one PDX in rank order of the LDA-based classifier score of metastatic efficiency, there was no pattern that could intuitively explain the score shift (FIG. 4A). This outcome was not too surprising given that much of the cell appearance is likely unrelated to metastasis-enabling functions, including the image signals associated with batch effects (FIG. S3).

To probe which features encapsulated in the latent cell descriptor are most discriminative of the metastatic state we first correlated each of the 56 features to the classifier score (FIG. 4B-4C). The correlations were calculated independently for each PDX using a classifier blind to the PDX (see FIG. 2A). For all 7 PDXs the last feature #56 stood out as highly negatively correlated to the classifier scores (FIG. 4C-4D). The correlation values fell outside the range of correlations observed for any other feature (FIG. 4E-4F). The distributions of values of feature #56 for individual cells clearly separated tumors with high versus low metastatic efficiency (FIG. 4G, 4H). However, as with the classifier score (FIG. 4A), a series of random cell snapshots from one PDX in rank order of feature #56 values did not reveal a cell image pattern that could intuitively explain the meaning of this feature (FIG. 4I). This suggests that feature #56 encoded a multifaceted image property reflecting the metastatic potential of melanoma PDXs that cannot readily be grasped by visual inspection.

Neither a series of cell images rank-ordered by classification scores of high versus low metastatic efficiency nor a series rank-ordered by feature #56 offered a visual clue as to which image properties may determine a cell's metastatic efficiency. We concluded that the natural variation of feature #56 values in our data may have been too low to give such clues, and/or that the natural variation of features unrelated to metastatic efficiency largely masked image shifts related to the variation of feature #56 between PDXs with low and high metastatic efficiency. To glean some of the image properties that are controlled by feature #56 we exploited the network decoder to generate a series of “in silico” cell images in which, given a particular location of a cell in the latent space, feature #56 was gradually altered while fixing all other features (FIG. 5A).

As expected, the changes in feature #56 negatively correlated with the changes they caused in the classifier score, regardless of the metastatic efficiency of the cells from which the images were derived (FIG. 5B). Autoencoder 110 brought a few advantages over previous attempts of visually interpreting latent features 110B′.

For example, autoencoder 110 allowed us to observe ‘pure’ image changes along a principal axis (e.g., manifestation of a single visual phenotype 150B) of metastatic efficiency change. In another example, autoencoder 110 allowed us to shift the value of feature #56 outside the value range of the natural distribution and thus to analyze the exaggerated cell images for emergent properties in cell appearance.

Upon morphing a PDX cell classified as low metastatic efficiency within a normalized z-score range for feature #56 of [−3.5, 3.5], we observed two properties emerging with the high metastatic efficiency domain. The formation of pseudopodal extensions and changes in the level of cellular light scattering as observed by brighter image intensities at the cell periphery and interior (FIG. 5C). The pseudopodal activity was visually best appreciated when compiling the morphing sequences into videos that shift a cell classified as low metastatic towards the high metastatic efficiency domain and, vice versa, a cell classified as highly metastatic towards the low metastatic efficiency domain.

Repeating the morphing for many PDX cells (FIG. S7) underscores pseudopod formation and enhanced light scattering as the systematic factors that distinguish cells with low feature #56 values/high metastatic efficiency from those with high feature #56 values/low metastatic efficiency. Moreover, by variation of all other latent space features one-by-one we visually confirmed this combination of morphological properties was specifically controlled by feature #56 (FIG. S8).

To corroborate our conclusion from synthetic images we tested whether “plastic” cells, which change their classifier score during the time course of acquisition from low to high efficiency or vice versa, displayed visually identifiable image transitions. First, we verified that temporal fluctuations in feature #56 negatively correlated with the temporal fluctuations in the classifier scores (FIG. 5D-5F). Second, we confirmed that PDX cells spontaneously transitioning from a predicted low to a predicted high metastatic efficiency displayed increased light scattering (FIG. 5G). We were not able to conclusively validate the enhanced protrusive activity in the time courses of experimental data. The subtlety and perhaps also the subcellular localization of this phenotype requires visualization outside the natural variation of the latent feature space.

When we applied the same feature-to-score correlation analysis to classifiers trained for discrimination of cell lines from PDXs, we found the three features #26, #27, and #36 as classification-driving (FIG. S9A-S9B). This result underscores two key properties of our interpretation of the latent space: First, distinct classification tasks are driven by different feature subsets in the latent space cell descriptor 110B, which capture distinguishing cell properties (e.g., visual phenotypes 150B). In all generality, the classification task is driven not by a single but by multiple latent space cell descriptors 110B. To enable interpretation of such multi-feature drivers, we generalized the traversal of the latent space by computing a trajectory that follows in every location the gradient of the classifier score. Since we used a linear LDA as an ML-based classifier 120, the gradient follows throughout the entire latent space the directions determined by the classifier coefficients (FIGS. S9C,S9D). Thus, we traversed the latent space 110BLS up and down in steps that are weighted by the LDA coefficients. For the classifier distinguishing PDXs from cell lines, the latent space 110BLS traversal to positions beyond the natural variation 110BNV in the data suggests that PDX cells exhibit a wider range of non-round morphologies than cell lines (FIG. S9E). However, for one cell the simulated PDX image outside the natural data range 110BNV displays an artefactual break-up of the cell volume, indicating an example of occasional failure of the described extrapolation strategy.

As a second test case, we trained another (e.g., unsupervised) adversarial autoencoder 110 (FIG. 1C) to capture an alternative latent space representation (e.g., latent feature vector 110B) of cell appearance. The autoencoder network training was performed on the same dataset of PDXs, cell lines, clones and melanocyte images as the first network, and was followed by training LDA classifiers to discriminate between high and low metastatic efficient PDXs, each blind to the PDX in test.

Because of the stochasticity in selecting mini-batches, the training converged to a different latent space cell image representation 110B. In this representation, several features 110B′, and not only feature 110B′ #56, correlated with the classifier score (FIG. S10A), as also reflected by multiple LDA 120 coefficients with high magnitudes (FIGS. S10B, S10C). Tracing PDX cells along the LDA 120 coefficients to latent space 110BLS locations outside the natural variation 110BNV of the data confirmed light scattering and pseudopodal extensions as the determinants between cells with classifications 120′ of high versus low metastatic efficiency, by shifting feature #56 in the latent representation determined by the original autoencoder network (compare FIG. S10D). These results establish the generalization of in silico latent features 110B′ amplification to higher-dimensional discriminant feature sets.

We were interested in the capacity of PDX-trained classifiers 120 to predict the spontaneous metastasis of tumor-forming melanoma cell line xenografts. We hypothesized that, despite the distinct morphologies of PDX and cell lines indicated by the classifier in FIGS. 2H and 2J, the core differentiating properties between low and high efficiency metastatic PDXs would be conserved for melanoma cell lines. Using the PDX-trained classifiers, A375, a BRAFV600E-mutated and NRAS wild-type melanoma cell line, originally excised from a primary malignant tumor was predicted as the most aggressive metastasizer (FIG. 6A). MV3, a BRAF wild-type and NRAS-mutated melanoma cell line, originally excised from a metastatic lymph node and described as highly metastatic, was predicted by the PDX-trained classifiers as the least aggressive (FIG. 6A).

Consistent with our previous analyses of the influence of the latent space features 110B′ on classification 120′, feature #56 was lower for A375 than for MV3 (FIG. 6B). We subcutaneously injected luciferase-labeled versions of A375 and MV3 cells into the flanks of NSG mice. Both cell models formed robust primary tumors at the site of injection (FIG. 6C-D) as well as metastases in the lungs and in multiple other remote organs (FIG. 6E-F).

Bioluminescence imaging of individual excised organs showed a higher spreading to organs other than the lungs in mice injected with A375 cells compared to those injected with MV3 cells (FIG. 6E-F). It was previously determined that the most robust measure of metastatic efficiency in this model was visually identifiable macrometastases in organs other than the lungs. As confirmation that the A375 cells metastasized more efficiently in this model, we found macrometastases in other organs in 5/5 mice xenografted with A375 cells versus in 1/5 mice xenografted with MV3 cells (FIG. 6G). Intriguingly, primary tumors in MV3-injected mice grew much faster than in A375-injected mice (FIG. 6H), in contrast to being less aggressive in spreading to remote organs, suggesting that primary tumor growth is uncoupled from the ability to produce remote d. Under the assumption that overall tumor burden would be limiting for metastatic dissemination instead of time after injection, we conclude, in agreement with the prediction of our classifier, that A375 cells are more metastatically efficient than MV3 cells in this model. Broadly, these data confirm that properties captured by the latent space cell descriptor define a specific gauge of the metastatic potential of melanoma that is independent of the tumorigenic potential.

Following initial diagnosis, it is standard practice for a melanoma biopsy to undergo mutational sequencing analysis to determine the best course of therapy. But, to our knowledge it has not been determined if there is a general mutational profile associated with more aggressively metastatic disease. While metastatic melanoma are expected to harbor a ‘standard’ set of primary mutations, such as those in BRAF or NRAS, and indeed all our PDX models and metastatic cell lines do contain an activating mutation in either one of these genes, we were curious as to whether secondary mutations in the genomic profiles of these cell models would encode information on the metastatic efficiency.

To address this question we examined the distributions of genomic distances among the PDX cell models and two cell lines vis-à-vis the distance distributions in the latent feature space. The conclusion from these experiments was that the states of oncogenic/likely-oncogenic mutations in the 20 most mutated genes in melanoma were insufficient for a prediction of the metastatic efficiency (FIG. S11). In fact, the oncogenic/likely-oncogenic mutations in the genes were not more predictive than non-oncogenic mutations or an unbiased analysis of a full panel of 1400 genes for metastatic states. Thus, image-based classifiers can identify more metastatically aggressive cancers, which is not currently possible for clinical diagnostics based on genomics.

Morphology has long been a cue for cell biologists and pathologists to recognize cell category and abnormalities related to disease. In this study, we rely on the exquisite sensitivity of deep learned artificial neural networks in recognizing subtle but systematic image patterns to classify different cell categories and cell states. To assess this potential we chose phase contrast light microscopy, an imaging modality that uses simple transmission of white or monochromatic light through an unlabeled cell specimen and thus minimizes experimental interference with the sensitive patient samples that we used in our study. A further advantage of phase contrast microscopy is that the imaging modality captures visually unstructured properties, which relate to a variety of cellular properties, including surface topography, organelle organization, cytoskeleton density and architecture, and interaction with fibrous extracellular matrix.

Our cell type classification 120′ may include the combination of an unsupervised deep learned autoencoder 110 for extraction of meaningful but visually hidden features (e.g., visual phenotypes 150B) followed by conventional supervised classifier 120 that may discriminate between distinct cell categories or classifications 120′.

The choice of this two-step implementation allowed us to construct several different cell classifiers 120 for different tasks using a one-time learned, common feature space. Thus, the task of distinguishing, for example, melanoma cell lines from normal melanocytes could benefit from the information extracted from PDXs, while PDXs could be divided into groups with high versus low metastatic propensity with the support of information extracted from melanoma cell lines and untransformed melanocytes.

Accordingly, sensitive classifiers 120 could be trained on relatively small data subsets—much smaller than would be required to train a deep-learned classifier for the same task. The approach is not only data-economical, but it greatly reduces computational costs as the deep learning procedure is performed only once on the full dataset. Indeed, in our study we learned a single latent feature space using time lapse sequences from over 12,000 cells (˜1.7 million snapshots); and then trained classifiers on data subsets that included labeled categories smaller than 1,000 cells. As an additional benefit of the orthogonalization of unsupervised feature extraction and supervised classifier training, we were able to evaluate the performance of our classifiers by repeated leave-one-out validation, verifying that the discriminative model training is completely independent of the cell type at test. A similar evaluation strategy, requiring the repeated re-training of a deep learned classifier, would likely become computationally prohibitive.

Among the cell classification tasks, we were able to distinguish the metastatic efficiency of stage III melanoma harvested from a xenotransplantation assay that had previously been shown to maintain the patient outcome. While the distinction was perfect at the level of PDXs, at the single cell level the classifier accuracy dropped to 70%. This is not necessarily a weakness of the classifier but speaks to the fact that tumor cells grown from a single cell clone are not homogeneous in function and/or appearance. Our estimates of classifier accuracy relies on leave-one-out strategies where the training set and the test set were completely non-overlapping, both with regards to the classified cell category and to the days the classified category was imaged. Thus, it can be assumed that the reported accuracies can be reproduced on new, independent PDXs.

Besides numerical testing, we validated the accuracy of our classifiers high versus low metastatic efficiency in a fully orthogonal experiment. We applied the PDX-trained classifiers to predict the metastatic efficiency of well-established melanoma cell lines and validated their predictions in mouse xenografts. We emphasize that the PDX-trained classifier has never encountered a cell line and that despite the significant differences between cell lines and PDXs (FIGS. 2H-2J), classifier 120 correctly predicted high metastatic potential for the cell line A375 and low potential for MV3 (FIG. 6).

Moreover, experimental results have shown, using in vivo barcoding as a readout for metastatic potential of cancer cell lines engrafted in mice, that A375 is more aggressive than SKMEL2, in agreement with our classifier's prediction (FIG. 6A). Intriguingly, the aggressiveness in primary tumor growth was reversed between A375 and MV3, supporting the notion that tumorigenesis and metastasis are unrelated phenomena (FIG. 6H). This shows that the latent feature space encodes cell properties that specifically contribute to cell functions required for metastatic spreading and that these features are orthogonal to features that distinguish cell lines from PDX models.

As elaborated herein, by exploiting the single cell variation of the latent feature space 110BLS occupancy and the associated variation in the scoring of a classifier 120 discriminating high from low metastatic melanoma, we identified latent features (e.g., feature #56) as predominant in prescribing metastatic propensity. Of note, the feature-to-classifier correlation analysis was not restricted to determining a single discriminatory feature (FIG. S9, S10) and was directly applicable to non-linear classifiers.

Visual inspection of cell images ranked by the classifier score 120′ for feature #56 did not reveal any salient cell image appearance that would distinguish efficiently from inefficiently metastasizing cells (FIGS. 4A,4I). These particular image properties were masked by cell appearances that are unrelated to the metastatic function. Moreover, the function-driving feature #56 represents a nonlinear combination of multiple image properties that are not readily discernible.

To test whether feature #56 encodes image properties that are human-interpretable but buried in the intrinsic heterogeneity of cell image appearances, we exploited the generative power of our autoencoder. We ‘shifted’ cells along the latent space axis of a specific feature (e.g., modifying a value of latent feature 110B′ #56) while leaving the other 55 latent feature 110B′ values fixed. The approach also allowed us to examine how cell appearances would change with feature #56 values outside the natural range 110BNV of our experimental data. Hence, the combination of purity and exaggeration allowed us to generate human discernible changes in image 30 appearance that correspond to a shift in metastatic efficiency classification 120′.

The experimental outcome of a single latent feature 110B′ (e.g., feature #56) driving the classification 120′ between two cell categories is by chance. As we show for the classification of PDXs versus cell lines, multiple latent features 110B′ may strongly correlate with the classifier score 120′. In this case, interpretation by visual inspection of exaggerated images 30 has to be achieved by traversing the latent space in trajectories that follow in every location the gradient of the classifier score. In the particular case of the LDA classifier 120, the gradient is spatially invariant and follows the combination of the LDA 120 coefficients. Thus, the proposed mechanism of visual latent space interpretation does not hinge on the identification of a single driving latent feature 110B′.

Once exaggerated in-silico images 30 offered a glimpse of key image properties (e.g., visual phenotypes 150B), distinguishing efficient from inefficient metastasizers, we could validate the predicted appearance shifts in experimental data. This was especially important to exclude the possibility that our extrapolation of feature values introduced image artifacts. We screened our data set for cells whose classification score and feature #56 values drifted from a low to high metastatic state or vice versa. We assumed that during such spontaneous dynamic events the variation in cell image appearances would be dominated, for a brief time window, by the variation in feature #56 and only marginally influenced by other features. Therefore, time-resolved data may present transitions in cell image appearance comparable to those induced by selective manipulation of latent space values along the direction of feature #56. It is highly unlikely to find a similarly pure transition between a pair of cells, explaining why we were unable to discern differences between cells with low and high metastatic efficiency in feature #56 ordered cell image series (FIG. 4A).

Analyses of appearance shifts in both exaggerated in silico images 30 and selected experimental images 20 unveiled cellular properties (e.g., visual phenotypes 150B) of highly metastatic melanoma. First, these cells seemed to form pseudopodal extensions (FIG. 5C, FIG. S7). Because of its subtlety, this phenotype was more difficult to discern visually during spontaneous transitions of cell states (FIG. 5G). Second, images of cells in a highly metastatic state displayed brighter cell peripheral and interior signals, indicative of alteration in cellular light scattering. Because light scattering affects the image signal globally, this phenotype was clearly apparent in simulations (FIG. 5C, FIG. S7) and in experimental time lapse sequences of transitions between cells states (FIG. 5G). Neither one of the two cell visual phenotypes 150B follows a mathematically intuitive formalism that could be implemented as an ab initio feature detector. This highlights the power of deep learned networks in extracting complex cell function-driving image appearances.

Pseudopodal extensions play critical roles in cell invasion and migration. However, at least in a simplified migration assay in tissue culture dishes, the highly metastatic cell population did not exhibit enhanced migration (FIG. S6). Recent work has suggested mechanistic links between enhanced branched actin formation in lamellipodial and enhanced cell cycle progression, especially in micro-metastases. Therefore, we offer as a hypothesis that the connection between pseudopod formation and metastatic efficiency predicted by our analysis relates to the lamellipodia-driven upregulation of proliferation and survival signals.

The observation that light scattering can indicate metastatic efficiency suggests that the cellular organelles and processes captured by light scattering are relevant to the metastatic process. Indeed, differences in light scattering upon acetic acid treatment are often used to detect cancerous cells in patients. Although the mechanisms underlying light scattering of cells are unclear, intracellular organelles such as phase separated droplets or lysosomes will be detected by changes to light scattering. With the establishment of our machine-learning based classifier, we are set to systematically probe the intersection of hypothetical metastasis-driving molecular processes, actual metastatic efficiency, and cell image appearance in follow-up studies.

We found that melanoma cell cultures derived from PDX tumors exhibited variable responses to traditional cell culture practices. Although some of the cell cultures retained high viability and proliferated readily, others exhibited extensive cell death and failed to proliferate. We determined that frequent media changes (<24 hours) and subculturing only at high (>50%) confluence dramatically increased the viability and proliferation of PDX-derived cell cultures. Although we observed no correlation between metastatic efficiency and robustness in cell culture, we followed these general cell culture practices for all PDX-derived cultures.

To create cell populations “cloned” from a single cell, cells were released from the culture dish via trypsinization and passed through a cell strainer to ensure single-cell solution, counted and then seeded on a 10 cm polystyrene tissue culture dish at low density of 350,000 cells/10 ml of phenol-red free DMEM. Single cells were identified via phase-contrast microscopy. The single cells were isolated using cloning rings and expanded within the ring. For clonal medium changes, the medium was aspirated within the cloning rings. Subsequently, conditioned medium from a culture dish with corresponding confluent cells were passed through a filter which removed any cells and cell debris and then added to each cloning ring. Once confluent within the cloning ring, the clonal populations were released via trypsinization inside the cloning ring, transferred to individual cell culture dishes, and allowed to expand until confluence.

We used three measures to assess metastatic efficiency. First, detection of BLI in the lungs. Second, detection of BLI in multiple organs beyond the lungs. Third, identification of “visceral metastasis”, macrometastases visually identifiable without BLI.

Targeted sequencing was performed on 6 out of 7 PDXs and the two cell lines A375 and MV3. Due to the difficulty in expanding the cells of PDX m528 in culture, we were not able to sequence this PDX. From the raw variant calling files, high confidence variants were determined by filtering variants found to have (a) strand bias, (b) depth of coverage <20 reads and alt allele frequency <20%. Common variants were filtered if they were in >1% allele frequency in any population. Oncogenic potential was assessed using oncokb-annotator.

Live cell phase contrast imaging was performed on a Nikon Ti microscope equipped with an environmental chamber held at 37° C. and 5% CO2 in 20× magnification (pixel size of 0.325 μm). In order to prevent morphological homogenization and to better mimic the collagenous ECM of the dermal stroma, we imaged cells on top of a thick slab of collagen. Collagen slabs were made from rat tail collagen Type 1 at a final concentration of 3 mg/mL, created by mixing with the appropriate volume of 10× PBS and water and neutralized with 1N NaOH. A total of 200 microliters (μL) of collagen solution was added to the glass bottom portion of a Gamma Irradiated 35 MM Glass Bottom Culture Dish. The dish was then placed in an incubator at 37° C. for 15 minutes to allow for polymerization.

Cells were seeded on top of the collagen slab at a final cell count of 5000 cells in 400 μL of medium per dish. This solution was carefully laid on top of the collagen slab, making sure not to disturb the collagen or spill any medium off of the collagen and onto the plastic of the dish. The dish was then placed in a 37° C. incubator for 4 hours. Following incubation, one mL of medium was gently added to the dish. The medium was gently stirred to suspend debris and unattached cells. The medium was then drawn off and gently replaced with two mL of fresh medium.

We took advantage of the observation that image regions associated with “cellular foreground” had lower temporal correlation than the background regions associated with the collagen slab because of their textured and dynamic nature. This allowed us to develop an image analysis pipeline that detected and tracked cells without segmenting the cell outline. This approach allowed us to deal with the vast variability in the appearance of the different cell models and batch imaging artifacts in the phase-contrast images. The detection was performed in super-pixels with a size equivalent to a 10×10 μm patch. For each patch in every image, we recorded two measurements, one temporal-dependent and the other intensity-dependent, generating two corresponding down sampled images reflecting the local probability of a cell being present. We used these as input to a particle tracking software, which detected and tracked local maxima of particularly high probability. The first measurement captures the patch's maximal spatial cross-correlation from frame t to frame t+1 within a search radius that can capture cell motion up to 60 μm/hour. The second measurement used the mean patch intensity in the raw image to capture the slightly brighter intensity of cells in relation to the background in phase-contrast imaging.

Notably, our reduced resolution in the segmentation-free detection and tracking approach would break for imaging in higher cell densities. A bounding box of 70×70 μm around each cell was defined and used for single cell segmentation and feature extraction. We excluded cells within 70 μm from the image boundaries to avoid analyzing cells entering or leaving the field of view and to avoid the characteristic uneven illumination in these regions. Tracking of single cells over 8 hours was performed manually using the default settings in CellTracker v1.1.

We have developed an unsupervised, generative representation for capturing cell image features using Adversarial Autoencoders (AAE) 110. The autoencoder 110 learns a compressed representation (e.g., latent feature vector 110B) of cell images 20 by encoding (e.g., encoder 110A) images 20 using a series of convolution and pooling layers leading ultimately to a lower dimensional embedding, or latent space 110BLS. Points in the embedding space 110BLS can then be decoded by a symmetric series of layers (e.g., decoder 110C) flowing in the opposite direction to reconstruct an image that, once trained, ideally appears nearly identical to the original input.

The training/optimization of AAE 110 is regularized (by using a second network during training) such that points close together in the embedding space will generate images sharing close visual resemblance/features. This convenient property can also generate synthetic/imaginary cell images 30 to interpolate the appearance of cells from different regions of the space. The adversarial component teaches the network to discriminate between features derived from real cells and those drawn randomly from the latent space 110BLS. We trained the regularized AAE 110 with bounding boxes of phase-contrast single cell images (of size 70 μm×70 μm, or 217×217 pixels) that were rescaled to 256×256 pixels. The AAE network 110 was trained to extract a 56-dimensional image encoding representation of cell appearance. This representation and its variation over time were used as descriptors for cell appearance and dynamics behaviour.

We verified that the 56-dimensional latent vector preserves a visual similarity measure for cell appearance, e.g., increasing distances between two data points in the latent space correspond to increasing differences between the input images. We first validated that variations in the latent vector 110B cause variations in cell appearances (FIG. S2A). To accomplish this we numerically perturbed the latent vector 110B after encoding a cell image 20 with varying amounts of noise and calculated the mean squared error between the raw and reconstructed images. As expected, the mean squared error between reconstructed and raw images monotonically increased with increasing amount of noise added in the latent space (FIG. S2B). Hence, the trained encoder generates a locally differentiable latent space 110BLS. Second, we interpolated a linear trajectory in the latent space between two experimentally observed cells, as well as between two random points, and confirmed, visually and quantitatively, that the decoded images gradually transform from one image to the other (FIG. S2C, S2D). Hence, the trained encoder 110A generates a latent space 110BLS without discontinuities. Third, we calculated the latent space distances between a cell at time t and the same cell at t+100 minutes and between a cell at time t and a neighboring cell in the same sample at time t. The distances between time-shifted latent space vectors for the same cell were shorter than those between neighboring cells (FIG. S2E). Hence, the combined effects of time variation in global imaging parameters and of morphological changes on displacements in the latent space tend to be smaller than the difference between cells.

In the case of the presented label-free imaging assay, batch effects may arise from uncontrolled experimental variables such as variations in the properties of the collagen gel, illumination artifacts, or inconsistencies in the phase ring alignment between sessions. Autoencoders are known to be very effective in capturing subtle image patterns. Therefore, they may pick up batch effects that mask image appearances related to the functional state of a cell. Under the assumption that intra-patient/cell line variability in image appearance is less than inter-patient/cell line appearance, we expect the latent cell descriptors of the same cell category on different days to be more similar than the descriptors of different cell categories imaged on the same day.

To test how strong batch effects may be in our data, we simultaneously imaged four different PDXs in an imaging session that we replicated on different days. Every cell was represented by the time-averaged latent space vector over the entire movie. We then computed the Euclidean distance as a measure of dissimilarity between descriptors from the same PDX imaged on different days to the distribution of Euclidean distances between different PDXs imaged on the same day (FIG. S3A). For three of the four tested PDXs we could not find a clear difference between the intra-PDX/inter-day similarity and the intra-day/inter-PDX similarity (FIG. S3B). Only PDX m610 displayed greater intra-PDX/inter-day similarity than intra-day/inter-PDX similarity.

Consistent with this assessment, visualization of all time-averaged cell descriptors over all PDXs and days using Principle Components Analysis (PCA) or t-distributed stochastic neighbor embedding (tSNE) projections neither showed cell clusters associated with different PDXs nor with different imaging days, except for m610 (FIG. S3C, S3D). These results suggest that the latent space cell descriptors are impacted by both experimental batch effects and putative differences in the functional states between PDXs.

To compare the performance of the deep-learned cell descriptors to conventional, shape-based descriptors of cell states we segmented phase contrast cell images of multiple cell types with diverse appearances.

Label-free cell segmentation is a challenging task, especially in the diverse landscape of shapes and appearance of the different melanoma cell systems we used. We used the Lineage Editing and Validation (LEVER) tool, a designated phase-contrast cell segmentation algorithm to segment single cells within the bounding boxes identified by the previously described segmentation-free cell tracking. Briefly, the LEVER segmentation is based on minimum cross entropy thresholding and additional post-processing. While the segmentation was not perfect, it generally performed robustly to cells from different origins and varied imaging conditions (FIG. S5A-B). We used MATLAB's function regionprops to extract 13 standard shape features from the segmentation masks produced by LEVER. These included: Area, MajorAxisLength, MinorAxisLength, Eccentricity, Orientation, ConvexArea, FilledArea, EulerNumber, EquivDiameter, Solidity, Extent, Perimeter, PerimeterOld.

We compared three different approaches to incorporating temporal information when using either the autoencoder-based representation or the shape-based representation of cell appearance (FIG. S5C). First, static snapshot images ignoring the temporal information. Second, averaging the cell static descriptors along a cell's trajectory, canceling noise for cells that do not undergo dramatic changes. Notably, the resulting cell descriptor matches the static descriptor in size and features. Accordingly, classifiers that were trained on average temporal descriptors could be applied to static snapshot descriptors (see FIGS. 4-5). In the third encoding we relied on the ‘bag of words’ (BOW) approach, in which each trajectory is represented by the distribution of discrete cell states, termed “code words.” A ‘dictionary’ of 100 code words was predetermined by k-means clustering on the full dataset of cell descriptors.

We found that purely shape-based descriptors could not distinguish cell lines from PDXs (FIG. 55D). This indicates that the autoencoder latent space captures information from the phase-contrast images that is missed by the shape features. Incorporation of temporal information, especially the time-averaging, slightly (but significantly) boosted the classification performance of LDA models derived from latent space cell descriptors (FIG. 55E). This outcome is consistent with computer vision studies concluding that explicit modeling of time may lead to only marginal gains in classification performance.

We used tSNE (FIG. S3C) and PCA (FIG. S3D) for dimensionality reduction. Each cell was represented by its time-averaged descriptors in the latent space. For tSNE we used a GPU-accelerated implementation.

We used MATLAB's vanilla implementation of Linear Discriminant Analysis (LDA) for the discrimination tasks (FIGS. 2-3) and to identify the cellular phenotypes that correlate with low or high metastatic efficiency (FIGS. 4, 5). The feature vector for each cell was given by the normalized latent cell descriptor extracted by the autoencoder. Normalization of each latent cell descriptor component (e.g., latent feature 110B′) to a z-score feature was accomplished as follows. The mean (μ) and standard deviation (σ) of a latent cell descriptor component were calculated across the full data set of cropped cell images and used to calculate the corresponding z-score measure: x_norm=(x−μ)/σ, i.e., the variation from the mean values in units of standard deviation that can later be compared across various different features.

For each classification task, the training data was kept completely separate from the testing data. Training and testing sets were assigned according to two methodologies. First, hold out all data from one cell type and train the classifier using all other cell types (FIG. 2A). Second, hold out all data from one cell type imaged in one day as the test set (“cell type—day”, e.g., FIG. 3F) and train the classifier on all other cell types excluding the data imaged on the same day as the test set (FIG. S4A). This second approach trained models that had never seen the cell type or data imaged on the same day of testing. In both classification settings we balanced the instances from each category for training by randomly selecting an equal number of observations from each class. This scheme was used for classification tasks involving categories containing more than one cell type: cell lines versus melanocytes, cell lines versus clonally expanded cell lines, cell lines versus PDXs, low versus high metastatic efficiency in PDXs (FIGS. 2-3). For statistical analysis, all the cells in a single test set are considered as a single independent observation. Hence, “cell type—day” testing sets provide more independent observations (N) at the cost of fewer cells imaged in each day compared to testing set of the form of “cell type”.

We used bootstrapping to statistically test the ability to predict metastatic efficiency from samples of 20 random cells. This was performed for “cell type” (FIG. 3D) or “cell type—day” (FIG. 3G) test sets. For each test set, we generated 1000 observations by repeatedly selecting 20 random cells (with repetitions), recorded the fraction of these cells that were classified as low efficiency and the 95% confidence interval of the median. Statistical significance in all settings was inferred using two statistical tests using each test set classifier's mean score: (1) The nonparametric Wilcoxon signed-rank test, considering the null hypothesis that the classifiers scores of observations from the two categories stem from the same distribution; (2) The Binomial test, considering the null hypothesis that the classifier prediction is random in respect to the ground truth labels. For inference of phenotypes that correlate with metastatic efficiency (FIG. 5) we used the classifier that was trained on the mean latent cell description along its trajectory (which proved to be superior to training with single snapshots) on latent cell descriptors derived from single snapshots, which hold the same, just noisier features.

The area under the Receiver Operating Characteristic (ROC) curve was recorded to assess and compare the discriminative accuracy of different tasks (FIGS. 2-3). The true-positive rate (TPR) or sensitivity is the percentage of “low” metastatic cells classified correctly. The false-positive rate (FPR) or (1-specificity) is the percent of “high” metastatic cells incorrectly classified as “low”. Area under the ROC curve (AUC) was used as a measure of discrimination power. Note that the scores of all cells from all relevant cell types were pooled together for this analysis. Different classifiers can produce different scores, which means that our analysis provides a lower bound (pessimistic estimation). ROC analysis could not be applied for individual (held-out) test sets because they consist of only a single ground truth label.

To generalize the in silico cell image amplification to multiple features, we traversed the high dimensional latent space 110BLS according to the corresponding LDA coefficients. More specifically, we moved up/down the classifier's score gradient by adding/subtracting multiples of one standard deviation of the unit vector weighted according to the LDA classifier coefficients.

We calculated a distance matrix to assess the similarity between all pairs of PDXs and the cell lines A375 and MV3. The distances were calculated in terms of the classifier score and of genomic mutation panels. m528 was excluded from the analysis due missing sequencing data (see above). For the distance matrix of the classifier score, we calculated the Jensen-Shannon (JS) divergence between the distributions of single cell classifier scores using the corresponding PDX-based classifiers. For the cell lines, a new classifier was trained using all cells from all seven PDXs. This classifier was used to determine the classifier score for A375 and MV3. For each cell type, the distribution was approximated with a 25 bin histogram. JS divergence was calculated on pairs of cell type classifier score distributions.

To calculate distance matrices based genomic mutations we considered three panels of established melanoma genomic mutation markers. Two genomic mutation panels were derived from variation of exomes associated with 1385 cancer-related genes (see above). Mutations in commonly mutated genes in melanoma were annotated using the Oncology Knowledgebase (OncoKB) and divided into (I) oncogenic or likely oncogenic (Table S3, FIG. S11B) and (ii) benign or unannotated (“non-oncogenic”) (Table S4, FIG. S11C). Mutational based genetic distances were derived by converting mutation scores to a binary state (1=presence, 0=absence) and computing the Jaccard index between cell types. In FIG. S11D we calculated distances using fast genome and metagenome distance estimation (MASH), which compared the K-mer profiles between samples, thus giving a distance of the raw sequence data, without biases introduced in the alignment and variant calling analysis.

The distance matrices derived from classifier scores and mutational states were correlated (Pearson correlation) to assess whether the genomic mutation state and image-derived classifier scores for low and high metastatic efficacies were linked.

For each classification task, the training data was kept completely separate from the testing data. For statistical analysis, all the cells in a single test set were considered as a single independent observation. We used bootstrapping to statistically test the ability to predict a category from samples of 20 random cells (FIG. 2D, FIG. 2G, FIG. 2J, FIG. 3D, FIG. 3G). Statistical significance in category classification of “cell type” (FIG. 2C, FIG. 2F, FIG. 2I, FIG. 3C) or “cell type—day” (FIG. 3F, FIG. S4C, FIG. S4E, FIG. S4G) was inferred using two statistical tests. (1) The nonparametric Wilcoxon signed-rank test, considering the null hypothesis that the classifiers scores of observations from the two categories stem from the same distribution; (2) The Binomial test, considering the null hypothesis that the classifier prediction is random in respect to the ground truth labels. The purpose of testing two different null hypotheses was to increase thoroughness, especially given the small sample sizes (number of cell types). Statistical significance of discrimination using cell shape and temporal information (FIG. S5D-F, FIG. S6A) was inferred using the Wilcoxon signed-rank test.

Embodiments of the invention may provide a practical application in the technological field of assistive diagnostics and screening of biological specimens, as elaborated herein. Embodiments of the invention may include a variety of improvements over currently available diagnostic methods and systems.

For example, system 100 may select specific latent features that significantly drive a classification of interest, to uncover underlying, or root causes of that classification.

In another example, system 100 may exaggerate manifestation of these specific latent features 110B′ far beyond a range of experimental data to identify weak visual signals in an input image, representing visual phenotypes 150B that are pertinent to the classification of interest.

In another example, by extracting the most prevalent latent features 110B′ of autoencoder 110 to represent depictions of biological specimens, system 100 may overcome the inherent difficulty imposed by lack of expert-provided annotations. For example, a physician may observe a multitude of cells, and only label or annotate a few of them as relevant for a classification of interest. However, system 100 may bypass the sparsity of labeled data by training autoencoder 110 using the full unlabeled data to produce robust latent representations 110B′, and then using these representations 110B′ to classify depictions of biological specimens according to a classification of interest.

In yet another example, system 100 may improve the computational process of classifying and/or screening of images (e.g., images depicting biological specimen), in relation to currently available classification methods: Autoencoder 110 only needs to be trained once on the available dataset to obtain the latent vector 110B representation. Therefore, specific classifiers that are adapted to produce specific classifications 120′ of interest may rely on this one-time obtained representation space, resulting in reduction of computational resource consumption (e.g., time, processing cycles, and the like).

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of screening biological specimens by at least one processor, the method comprising:
receiving a sample image depicting a biological specimen;
applying a machine-learning (ML) based autoencoder model on the sample image, wherein said autoencoder model is trained to generate a reconstructed version of the sample image, via a latent feature vector;
associating at least one latent feature of the latent feature vector to at least one corresponding visual phenotype of the biological specimen; and
screening the biological specimen based on said association.

2. The method of claim 1, further comprising for at least one latent feature:
modifying a value of the latent feature to produce a vector set, comprising a plurality of latent feature vectors;
applying a decoder portion of the autoencoder on the vector set, to produce a corresponding reconstructed image set, representing evolution of a visual phenotype of the biological specimen; and
associating the latent feature to the visual phenotype based on the reconstructed image set.

3. The method of claim 2, further comprising for the at least one latent feature:
applying a first ML-based classification model on one or more latent feature vectors of the vector set, to calculate corresponding classification scores, wherein each classification score represents pertinence of a relevant latent feature vector to a predefined class; and
associating the latent feature to the visual phenotype further based on the one or more classification scores.

4. The method of claim 3, further comprising for the at least one latent feature:
based on the classification scores, attributing a significance score to the at least one latent feature, wherein said significance score represents significance of the at least one latent feature in driving an outcome of the first ML-based classification model; and
associating the latent feature to the visual phenotype further based on the significance score.

5. The method of claim 2, wherein modifying a value of at least one latent feature comprises:
for one or more latent features, determining a range and a step size, so as to define a latent feature space of the autoencoder model; and
modifying the value of the one or more latent features so as to traverse through the latent space of the autoencoder model.

6. The method of claim 4, further comprising:
selecting one or more latent features based on their attributed significance scores;
calculating a trajectory that follows a gradient of the classification score based on the selected one or more latent features; and
modifying the value of the one or more selected latent features according to the calculated trajectory, so as to traverse through a latent space of the autoencoder model.

7. The method of claim 2, wherein modifying a value of at least one latent feature comprises:
accumulating a plurality of original values of the latent feature, corresponding to a plurality of sample images;
calculating a natural variation of a latent feature space, defined by the latent feature vector; and
modifying the value of the latent feature beyond the calculated natural variation of the latent feature space.

8. The method of claim 1, further comprising:
applying the autoencoder model on a sample image depicting a biological specimen, to produce a corresponding reconstructed version of the sample image;
calculating a first loss function value, based on comparison between the sample image and reconstructed image; and
training the autoencoder model so as to minimize said first loss function value.

9. The method of claim 8, further comprising:
providing a second ML-based classification model, trained to calculate a classification score that represents pertinence of a biological specimen depicted in an image to a predefined class;
applying the second classification model on the sample image to produce a first classification score;
applying the second classification model on the reconstructed version of the sample image to produce a second classification score; and
training the autoencoder model further based on the first classification score and second classification score.

10. The method of claim 8, further comprising:
producing a second loss function value, representing discrepancy between a classification score of the sample image and a classification score of the reconstructed image; and
training the autoencoder model further based on the second loss function value.

11. The method of claim 1, wherein screening biological specimen is selected from a list consisting of providing diagnostic evaluation of an underlying cause that contributes the classification score, based on the visual phenotype of the biological specimen; performing triage of a biological specimen, based on a visual phenotype of the biological specimen; and applying a treatment agent, based on the visual phenotype of the biological specimen.

12. The method of claim 2, wherein the visual phenotype of the biological specimen is undetectable by a human observer in the sample image, and wherein evolution of the visual phenotype comprises amplification of the visual phenotype, such that the visual phenotype is detectable by the human observer in at least one reconstructed image of the reconstructed image set.

13. A system for screening biological specimens by at least one processor, the system comprising: a non-transitory memory device, wherein modules of instruction code are stored, and a processor associated with the memory device, and configured to execute the modules of instruction code, whereupon execution of said modules of instruction code, the at least one processor is configured to:
receive a sample image depicting a biological specimen;
apply a machine-learning (ML) based autoencoder model on the sample image, wherein said autoencoder model is trained to generate a reconstructed version of the sample image, via a latent feature vector;
associate at least one latent feature of the latent feature vector to at least one corresponding visual phenotype of the biological specimen; and
screen the biological specimen based on said association.

14. The system of claim 13, wherein the at least one processor is further configured, for at least one latent feature, to:
modify a value of the latent feature to produce a vector set, comprising a plurality of latent feature vectors;
apply a decoder portion of the autoencoder on the vector set, to produce a corresponding reconstructed image set, representing evolution of a visual phenotype of the biological specimen; and
associate the latent feature to the visual phenotype based on the reconstructed image set.

15. The system of claim 14, wherein the at least one processor is further configured, for the at least one latent feature, to:
apply a first ML-based classification model on one or more latent feature vectors of the vector set, to calculate corresponding classification scores, wherein each classification score represents pertinence of a relevant latent feature vector to a predefined class; and
associate the latent feature to the visual phenotype further based on the one or more classification scores.

16. The system of claim 15, wherein the at least one processor is further configured, for the at least one latent feature, to:
attribute a significance score to the at least one latent feature based on the classification scores, wherein said significance score represents significance of the at least one latent feature in driving an outcome of the first ML-based classification model; and
associate the latent feature to the visual phenotype further based on the significance score.

17. The system of claim 16, wherein the at least one processor is further configured to:
selecting one or more latent features based on their attributed significance scores;
calculating a trajectory that follows a gradient of the classification score based on the selected one or more latent features; and
modifying the value of the one or more selected latent features according to the calculated trajectory, so as to traverse through a latent space of the autoencoder model.

18. The system of claim 14, wherein the at least one processor is configured to modify a value of at least one latent feature by:
accumulating a plurality of original values of the latent feature, corresponding to a plurality of sample images;
calculating a natural variation of a latent feature space, defined by the latent feature vector; and
modifying the value of the latent feature beyond the calculated natural variation of the latent feature space.

19. The system of claim 13, wherein screening biological specimen is selected from a list consisting of providing diagnostic evaluation of an underlying cause that contributes the classification score, based on the visual phenotype of the biological specimen; performing triage of a biological specimen, based on a visual phenotype of the biological specimen; and applying a treatment agent, based on the visual phenotype of the biological specimen.
